(19) United States
(12) Patent Application Publication
(10) Pub. No.: US 2026/0335067 A1
(43) Pub. Date: Oct. 29, 2026

(54) INTRAOCULAR LENS SIMULATOR

(71) Applicant: Alcon Inc., Fribourg (CH)

(72) Inventors: Sebastian REQUENA, Crowley, TX (US); William Arthur RAIFORD, III, Fort Worth, TX (US); Sophia IPPOLITO, Fort Worth, TX (US); Himansu Shekhar PATTANAIK, Fort Worth, TX (US); Deanna MOSCHITTA, Fort Worth, TX (US); Shinwook LEE, Arlington, TX (US); Brian Craig COX, Mansfield, TX (US); Philip ROSE, Fort Worth, TX (US); Guillermo Marcos PEREZ SANCHEZ, Fort Worth, TX (US)

(21) Appl. No.: 19/657,239

(22) Filed: Apr. 24, 2026

Related U.S. Application Data
(60) Provisional application No. 63/794,970, filed on Apr. 25, 2025.

(51) Int. Cl. A61F 2/16; A61B 3/113

(52) U.S. Cl. CPC A61F 2/16; A61B 3/113; A61F 2240/008

(57) ABSTRACT

The present disclosure generally relates to techniques for simulating intraocular lens (IOL) implantation in a patient. In one embodiment, an optical system includes a support structure and a model eye including an artificial cornea coupled to the support structure and disposed along an optical axis of the optical system. The model eye further includes an IOL assembly configured to be removably coupled to the support structure. In a coupled configuration, the IOL assembly is disposed proximally to the artificial cornea and aligned with the optical axis. The optical system further includes an eyepiece assembly disposed proximally to the model eye and aligned with the optical axis. Advantageously the optical system provides controlled testing of vision under different conditions and distances, dynamic focusing capability, objective assessment of visual disturbances and lens design updates of IOL implantation before surgery, and improved efficiency for evaluation time and patient feedback.

### CROSS-REFERENCE TO RELATED APPLICATIONS

This application claims the benefit of U.S. Provisional Application No. 63/794,970, filed Apr. 25, 2025, which is incorporated herein by reference in its entirety.

### FIELD

The present disclosure relates to ophthalmic apparatuses, systems, and methods, for example, intraocular lens (IOL) simulator apparatuses, systems, and methods for simulating IOL implantation in a patient.

### BACKGROUND

IOLs are implanted in patients’ eyes to address a number of medical conditions, such as cataracts, nearsightedness, farsightedness, astigmatism, and presbyopia. IOL technology continues to develop, and some recent advancements include extended depth of focus (EDOF) lenses, multifocal lenses having three or more focal points, light-adjustable lenses, lenses for correction of spherical aberrations, blue-light and violet-light filtering lenses, and so forth.

Bringing new IOL designs to market requires substantial resources, including the completion of several phases of clinical trials to obtain regulatory approval. During the clinical trials, the new IOLs are implanted and their performance compared with IOLs of existing designs. However, there are some challenges to assessing new IOL designs. The clinical trials can be lengthy, often extending 6-12 months or longer. The performance of the IOLs depends on the quality of the implantation of the IOLs, which can vary with the skill of the ophthalmologist. The reported performance of the IOLs depends primarily on patient feedback, but patient satisfaction is highly subjective and also can vary. There is no standardized approach for objectively describing visual disturbances such as halos and starbursts. Further, the incidence of the visual disturbances can be subtle enough that patients do not report them unless prompted. Many advanced features of IOLs (e.g., multifocal, EDOF) rely on neuroadaptation (e.g., the brain adapting to the change in optics), which can be difficult to predict and difficult to measure during a clinical trial.

### SUMMARY

Accordingly, there is a need to develop an IOL simulator that provides controlled testing of vision under different conditions (e.g., photopic, mesopic, scotopic) as well as different distances (e.g., using adjustable targets and displays) to simulate IOL implantation before surgery. Further, there is a need to develop an IOL simulator that enables virtual evaluation and comparison of different IOL designs (e.g., for each patient eye) before surgery. Further, there is a need to develop an IOL simulator that provides dynamic focusing capability (e.g., real time adjustments) using a liquid lens and a model eye with an artificial cornea and interchangeable IOL assemblies. Further, there is a need to develop an IOL simulator that uses objective assessment of visual disturbances and lens design updates of IOL implantation before surgery. Further, there is a need to develop an IOL simulator that provides improved efficiency for evaluation time and patient feedback.

This novel approach provides different IOL designs for patients to experience before surgery (e.g., improved patient feedback), reduces risk of post-implantation issues (e.g., photic phenomena), facilitates better identification of visual disturbances (e.g., halo, starbursts, photic phenomena), supports accurate IOL design updates, reduces evaluation time relative to traditional clinical trials, and reduces reliance on subject patient feedback in favor of objective assessment (e.g., horizontal error, vergence error, vertical error, left viewfinder focus score, left viewfinder focus position, right viewfinder focus score, right viewfinder focus position).

Embodiments of the present disclosure generally relate to techniques for simulating IOL implantation in a patient. In some embodiments, an optical system includes a support structure and a model eye including an artificial cornea coupled to the support structure and disposed along an optical axis of the optical system. The model eye further includes an IOL assembly configured to be removably coupled to the support structure. In a coupled configuration, the IOL assembly is disposed proximally to the artificial cornea and aligned with the optical axis. The optical system further includes an eyepiece assembly disposed proximally to the model eye and aligned with the optical axis.

The techniques described herein enable IOLs to be easily interchanged in preclinical evaluation procedures, allowing patients and medical providers to evaluate different designs and/or combinations of designs before committing to implantation of the IOLs. The improved evaluation capability reduces a likelihood of dissatisfaction following the implantation. The techniques described herein enable patients to provide more informative, more responsive feedback during the preclinical evaluation, which may include better identification of visual disturbances. The improved patient feedback supports more responsive and more accurate updates to the IOL designs.

In some embodiments, an optical system may include a support structure, a model eye, and an eyepiece assembly. In some embodiments, the model eye may be disposed along an optical axis of the optical system. In some embodiments, the model eye may include an artificial cornea coupled to the support structure and disposed along the optical axis of the optical system. In some embodiments, the model eye may include an intraocular lens (IOL) assembly configured to be removably coupled to the support structure. In some embodiments, in a coupled configuration, the IOL assembly may be disposed proximally to the artificial cornea and aligned with the optical axis. In some embodiments, the eyepiece assembly may be disposed proximally to the model eye and aligned with the optical axis. In some embodiments, the optical system may be configured to simulate vision of a patient after an IOL implantation.

In some embodiments, the optical system may further include a liquid lens. In some embodiments, the liquid lens may be disposed distally to the artificial cornea and aligned with the optical axis. In some embodiments, the optical system may further include a controller. In some embodiments, the controller may be communicatively coupled to the liquid lens. In some embodiments, the controller may be configured to transmit a control signal to control a shape of the liquid lens.

In some embodiments, the IOL assembly may include a body and an IOL wet cell. In some embodiments, the body may define an exterior surface, a handle extending from the exterior surface, and an interior volume. In some embodiments, the IOL wet cell may be disposed within the interior volume. In some embodiments, the body may further define a recess from a distal surface. In some embodiments, the IOL assembly may further include an aperture plate. In some embodiments, the aperture plate may be configured to be retained in the recess. In some embodiments, the aperture plate may be aligned with the optical axis in a retained configuration.

In some embodiments, the model eye may further include a base. In some embodiments, the base may define a proximal end opening and a distal end opening. In some embodiments, the artificial cornea may be disposed in the distal end opening. In some embodiments, the base may define a first circumferential opening. In some embodiments, the first circumferential opening may be configured to receive the IOL assembly into the base. In some embodiments, the base may further define a second circumferential opening. In some embodiments, the model eye may further include a first alignment component. In some embodiments, the first alignment component may be configured to be received into the second circumferential opening. In some embodiments, the IOL assembly may be aligned with the optical axis when the exterior surface of the body contacts the first alignment component.

In some embodiments, the optical system may further include a second alignment component coupled to the support structure. In some embodiments, the artificial cornea may be aligned with the optical axis when an exterior surface of the base contacts the second alignment component. In some embodiments, the first alignment component may contact an upper surface of the second alignment component.

In some embodiments, the IOL assembly may further include an identifier. In some embodiments, the optical system may further include a controller. In some embodiments, the controller may be configured to detect the identifier when the IOL assembly is in the coupled configuration. In some embodiments, the controller may be configured to apply, based on the identifier, one or more settings for a preclinical evaluation procedure that simulates vision of a patient after performing an IOL implantation. In some embodiments, the controller may be further configured to receive, by one or more input devices, indications of visual disturbances experienced by the patient during the preclinical evaluation procedure.

In some embodiments, the eyepiece assembly may include a first lens, a beamsplitter, and a second lens. In some embodiments, the beamsplitter may be disposed proximally to the first lens. In some embodiments, the second lens may be disposed proximally to the beamsplitter. In some embodiments, the optical system may further include a viewfinder camera. In some embodiments, the beamsplitter may be configured to reflect a portion of light incident on the model eye toward the viewfinder camera.

In some embodiments, the optical system may further include a pupil camera. In some embodiments, the beamsplitter may be further configured to reflect a portion of light incident on the second lens toward the pupil camera. In some embodiments, the optical system may further include an infrared light source oriented toward an eye of a patient. In some embodiments, the light incident on the second lens may include infrared light reflected from the eye. In some embodiments, the optical system may further include a controller. In some embodiments, the controller may be configured to track a position of a pupil of the eye. In some embodiments, the controller may be configured to generate, based on the position of the pupil, a visual acuity curve for the eye.

In some embodiments, a method of simulating intraocular lens (IOL) implantation may include configuring an optical simulation system by removably coupling an IOL assembly within a model eye aligned along an optical axis of the optical simulation system. In some embodiments, the method may further include positioning a patient to view a target through the model eye. In some embodiments, the method may further include presenting visual reference information as the target and acquiring patient response data while the patient views the target through the model eye. In some embodiments, the method may further include generating an assessment output based on the acquired patient response data, the assessment output characterizing visual performance associated with the IOL assembly.

In some embodiments, removably coupling the IOL assembly may include selecting one of a plurality of interchangeable IOL assemblies each providing a different optical characteristic.

In some embodiments, presenting the visual reference information may include displaying optotypes, contrast patterns, defocus targets, simulated night driving scenes, or a combination thereof at a simulated distance.

In some embodiments, acquiring patient response data may include receiving user input indicative of visual acuity, contrast sensitivity, subjective visual quality, or a combination thereof.

In some embodiments, generating the assessment output may include generating a visual acuity curve corresponding to the IOL assembly.

In some embodiments, the method may further include storing information indicative of one or more visual disturbances experienced by the patient while viewing the target through the model eye. In some embodiments, the one or more visual disturbances may include glare, halos, starbursts, reduced contrast, depth of focus artifacts, or a combination thereof.

In some embodiments, the method may further include repeating steps (1) through (4) of the method for a second IOL assembly. In some embodiments, the second IOL assembly may have at least one different optical characteristic than the IOL assembly. In some embodiments, the method may further include comparing assessment outputs between the IOL assembly and the second IOL assembly.

In some embodiments, the visual reference information and the assessment output may be based on received information identifying the IOL assembly, a vision assessment procedure, or both.

Although ophthalmic procedures are discussed as examples of procedures that may benefit from the described embodiments, the advantages of the surgical devices and systems described herein may benefit procedures in other clinical and/or surgical environments as well.

Implementations of any of the techniques described above may include a system, a method, a process, a device, and/or an apparatus. The details of one or more implementations are set forth in the accompanying drawings and the description below. Other features will be apparent from the description and drawings, and from the claims.

Further features and example embodiments of the present disclosure, as well as the structure and operation of various embodiments, are described in detail below with reference to the accompanying drawings. It is noted that the embodiments are not limited to the specific embodiments described herein. Such embodiments are presented herein for illustrative purposes only. Additional embodiments will be apparent to persons skilled in the relevant art(s) based on the teachings contained herein.

### BRIEF DESCRIPTION OF THE DRAWINGS/FIGURES

The accompanying drawings, which are incorporated herein and form part of the specification, illustrate the embodiments and, together with the description, further serve to explain the principles of the embodiments and to enable a person skilled in the relevant art(s) to make and use the embodiments.

FIG. 1A is a perspective view of a portion of an example optical system, according to an example embodiment.

FIG. 1B is a distant perspective view of the example optical system shown in FIG. 1A, according to an example embodiment.

FIG. 2 illustrates a block diagram of the example optical system shown in FIGS. 1A and 1B, according to an example embodiment.

FIG. 3A is a perspective view of a distal end of an example IOL simulator device, according to an example embodiment.

FIG. 3B is a perspective view of a proximal end of a monocular subassembly of the example IOL simulator device shown in FIG. 3A, according to an example embodiment.

FIG. 4A is a top cross-sectional view of the example IOL simulator device shown in FIG. 3A, according to an example embodiment.

FIG. 4B is a side cross-sectional view of the example IOL simulator device shown in FIG. 3A, according to an example embodiment.

FIG. 5 is an exploded view of an example model eye with removable IOL assembly, according to an example embodiment.

FIG. 6 illustrates a system diagram of an example IOL simulator system, according to an example embodiment.

FIG. 7 shows a viewfinder display for a preclinical evaluation procedure using the example IOL simulator system shown in FIG. 6, according to an example embodiment.

FIG. 8 shows a viewfinder display for a preclinical evaluation procedure using the example IOL simulator system shown in FIG. 6, according to an example embodiment.

FIG. 9 shows a pupil display for a preclinical evaluation procedure using the example IOL simulator system shown in FIG. 6, according to an example embodiment.

FIG. 10 illustrates a flow diagram for simulating IOL implantation, according to an example embodiment.

The features and example embodiments of the present disclosure will become more apparent from the detailed description set forth below when taken in conjunction with the drawings, in which like reference characters identify corresponding elements throughout. In the drawings, like reference numbers generally indicate identical, functionally similar, and/or structurally similar elements. Additionally, generally, the left-most digit(s) of a reference number identifies the drawing in which the reference number first appears. Unless otherwise indicated, the drawings provided throughout the disclosure should not be interpreted as to-scale drawings.

### DETAILED DESCRIPTION

Provided herein are system, apparatus, device, method, and/or computer program product embodiments, and/or combinations and sub-combinations thereof, for ophthalmic systems, such as an IOL simulator for simulating IOL implantation in a patient.

An IOL simulator as described below includes a support structure, a model eye having an artificial cornea and an IOL assembly, an eyepiece assembly, and a controller, where the controller applies one or more settings for a preclinical evaluation procedure for vision of a patient after a simulated IOL implantation.

A method of simulating IOL implantation as described below includes removably coupling an IOL assembly within a model eye aligned along an optical axis of an IOL simulator, positioning a patient to view a target through the model eye, presenting visual reference information as the target and acquiring patient response data while the patient views the target through the model eye, and generating an assessment output based on the acquired patient response data, where the assessment output characterizes visual performance associated with the IOL assembly.

This specification discloses one or more embodiments that incorporate the features of this present disclosure.

The embodiment(s) described, and references in the specification to “one embodiment,” “an embodiment,” “an example embodiment,” “an exemplary embodiment,” etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is understood that it is within the knowledge of one skilled in the art to effect such feature, structure, or characteristic in connection with other embodiments whether or not explicitly described.

Spatially relative terms, such as “beneath,” “below,” “lower,” “above,” “on,” “upper” and the like, may be used herein for ease of description to describe one element or feature’s relationship to another element(s) or feature(s) as illustrated in the figures. The spatially relative terms are intended to encompass different orientations of the device in use or operation in addition to the orientation depicted in the figures. The apparatus may be otherwise oriented (rotated 90 degrees or at other orientations) and the spatially relative descriptors used herein may likewise be interpreted accordingly.

Numerical values, including endpoints of ranges, can be expressed herein as approximations preceded by the term “about,” “substantially,” “approximately,” or the like. In such cases, other embodiments include the particular numerical value. Regardless of whether a numerical value is expressed as an approximation, two embodiments are included in this disclosure: one expressed as an approximation, and another not expressed as an approximation. It will be further understood that an endpoint of each range is significant both in relation to another endpoint, and independently of another endpoint.

Embodiments of the disclosure may be implemented in hardware, firmware, software, or any combination thereof. Embodiments of the disclosure may also be implemented as instructions stored on a machine-readable medium, which may be read and executed by one or more processors. A machine-readable medium may include any mechanism for storing or transmitting information in a form readable by a machine (e.g., a computing device). For example, a machine-readable medium may include read only memory (ROM); random access memory (RAM); magnetic disk storage media; optical storage media; flash memory devices; electrical, optical, acoustical or other forms of propagated signals (e.g., carrier waves, infrared signals, digital signals, etc.), and others. Further, firmware, software, routines, and/or instructions may be described herein as performing certain actions. However, it should be appreciated that such descriptions are merely for convenience and that such actions in fact result from computing devices, processors, controllers, or other devices executing the firmware, software, routines, instructions, etc.

The term “distance vision” as used herein indicates vision for objects that are at a calibrated distance from the viewer, for example, at least 20 feet or more from the viewer. The distance vision is for objects farther than the intermediate vision.

The term “intermediate vision” as used herein indicates vision for objects that are at a calibrated distance from the viewer, for example, between 18 inches and 20 feet from the viewer. The intermediate vision is for objects farther than the near vision.

The term “near vision” as used herein indicates vision for objects that are at a calibrated distance from the viewer, for example, 18 inches or less from the viewer. The near vision is for objects closer than the intermediate vision.

The term “photopic” or “photopic vision” as used herein indicates vision of the eye under well-lit conditions, for example, color vision in bright light or daylight conditions (e.g., luminance levels from 10 to 10⁸ cd/m²). In some embodiments, photopic light may have a central wavelength (peak sensitivity) of about 555 nm.

The term “mesopic” or “mesopic vision” as used herein indicates vision of the eye under low-light conditions, for example, color vision in dusk/dawn light or twilight conditions (e.g., luminance levels from 10⁻³ to 10 cd/m²). In some embodiments, mesopic light may have a central wavelength (peak sensitivity) of about 515 nm.

The term “scotopic” or “scotopic vision” as used herein indicates vision of the eye under very low-light conditions, for example, monochromic vision in nighttime or darkness conditions (e.g., luminance levels from 10⁻⁶ to 10⁻³ cd/m²). In some embodiments, scotopic light may have a central wavelength (peak sensitivity) of about 507 nm.

The term “visual acuity” or “VA” as used herein indicates a measurement of the clarity of vision. In some embodiments, visual acuity can characterize near vision, intermediate vision, and/or distance vision. Visual acuity (VA) can measure how well a viewer’s eyes can distinguish shapes at a given distance via an eye exam (e.g., Snellen chart, LogMAR chart). A Snellen chart measures visual acuity in a distance (foot) scale expressed as a ratio (e.g., 20/20, 20/25), where the numerator signifies the distance between the viewer and the chart (e.g., 20 feet), and the denominator signifies the distance at which a person with normal vision can read the same line (e.g., 20/20 is normal vision, 20/25 means the viewer can see at 20 feet what a person with normal vision can see at 25 feet). Visual acuity can be measured in different scales (e.g., foot scale, meter scale, LogMAR scale). In some embodiments, the visual acuity of a lens (e.g., an IOL) may be simulated through a simulated environment (e.g., simulated binocular VA), for example, using the American National Standards Institute (ANSI) method that provides a computer-generated test that mimics a real-life visual acuity test based on a patient’s pupil size (e.g., 3.0 mm diameter).

The term “logarithm of the minimum angle of resolution” or “LogMAR” as used herein indicates a chart consisting of rows of letters to estimate visual acuity. The LogMAR chart assesses visual acuity by scoring how many letters a viewer gets wrong, with each incorrect letter on a line adding 0.02 log units to the score. The LogMAR score can be correlated to the foot score (e.g., 20/20 corresponds to 0 log units, 20/25 corresponds to 0.1 log units, 20/32 corresponds to 0.2 log units, etc.).

The present disclosure generally relates to techniques for simulating IOL implantation in a patient. In some embodiments, an optical system includes a support structure and a model eye including an artificial cornea coupled to the support structure and disposed along an optical axis of the optical system. The model eye further includes an IOL assembly configured to be removably coupled to the support structure. In a coupled configuration, the IOL assembly is disposed proximally to the artificial cornea and aligned with the optical axis. The optical system further includes an eyepiece assembly disposed proximally to the model eye and aligned with the optical axis.

The techniques described herein enable IOLs to be easily interchanged in preclinical evaluation procedures, allowing patients and medical providers to evaluate different designs and/or combinations of designs before committing to implantation of the IOLs. The improved evaluation capability provides a reduced likelihood of dissatisfaction following the implantation. The techniques described herein enable patients to provide more informative, more responsive feedback during the evaluation, which may include better identification of visual disturbances. The improved feedback provided will support more responsive and more accurate updates to the IOL designs.

As discussed above, a problem in the art is that current methods for evaluating new IOL designs present technical and operational limitations that may limit speed and efficiency of achieving repeatable and timely characterization of IOL performance and patient experience. First, clinical trials are often lengthy (e.g., 6-12 months or more) and require substantial resources before IOL design decisions can be validated. Second, trial outcomes may vary due to differences in surgical technique during the IOL implantation procedure. Third, clinical evaluation frequently relies on subjective patient feedback, which lacks standardization, and visual disturbances (e.g., halos, starbursts, photic phenomena) are difficult to quantify and often are not reported. Fourth, many advanced IOL features (e.g., multifocal, EDOF) rely on neuroadaptation, which is unpredictable and difficult to measure during trials.

In order to address this problem, the systems and methods of the present disclosure provide an optical system (e.g., IOL simulator) for simulating IOL implantation before surgery. The described optical system includes a model eye with an artificial cornea (e.g., to mimic a patient’s cornea) and an interchangeable IOL assembly (e.g., to mimic a patient’s planned IOL for implantation) to simulate vision of the patient after IOL implantation and conduct a preclinical evaluation process. Also, the described optical system include a variable-focus optical element (e.g., a liquid lens) for focal adjustments in real time. This provides an improvement to IOL design technology by enabling controlled testing of vision for a simulated IOL implantation under different conditions (e.g., photopic, mesopic, scotopic) and different distances using adjustable targets and displays. This also provides an improvement to IOL design technology by providing a repeatable, controllable, and clinically relevant optical evaluation technique (IOL simulation) that shortens patient feedback cycles, reduces dependence on surgical variability, and enables standardized (objective) assessment of both visual acuity and visual disturbances before surgery.

The disclosed systems and methods provide a practical application in the field of ophthalmic or optometric instrumentation by improving how IOL performance is evaluated and selected through a specific, engineered optical pathway and adjustable measurement framework. The optical system physically simulates IOL implantation optics using an artificial cornea and a removable IOL assembly aligned on an optical axis and viewed through an eyepiece assembly, thereby enabling real-world, vision evaluation prior to surgery. This produces concrete technical effects including standardized, repeatable IOL assessment, reduced confounding from surgical variability, improved identification and documentation of visual disturbances, and accelerated evaluation cycles.

Embodiments of IOL simulator apparatuses, systems, and methods as discussed below provide controlled testing of vision under different conditions as well as different distances to simulate IOL implantation before surgery.

Further, embodiments of IOL simulator apparatuses, systems, and methods as discussed below provide virtual evaluation and comparison of different IOL designs before surgery.

Further, embodiments of IOL simulator apparatuses, systems, and methods as discussed below provide dynamic focusing capability using a liquid lens and a model eye with an artificial cornea and interchangeable IOL assemblies.

Further, embodiments of IOL simulator apparatuses, systems, and methods as discussed below provide objective assessment of visual disturbances and lens design updates of IOL implantation before surgery.

Further, embodiments of IOL simulator apparatuses, systems, and methods as discussed below provide improved efficiency for evaluation time and patient feedback.

This novel approach provides different IOL designs for a patient to experience in order to optimize an IOL design before surgery.

Further, this novel approach reduces risk of post-implantation issues.

Further, this novel approach facilitates better identification of visual disturbances.

Further, this novel approach supports accurate IOL design updates.

Further, this novel approach reduces evaluation time relative to traditional clinical trials.

Further, this novel approach reduces reliance on subject patient feedback in favor of objective assessment.

### Example Optical System

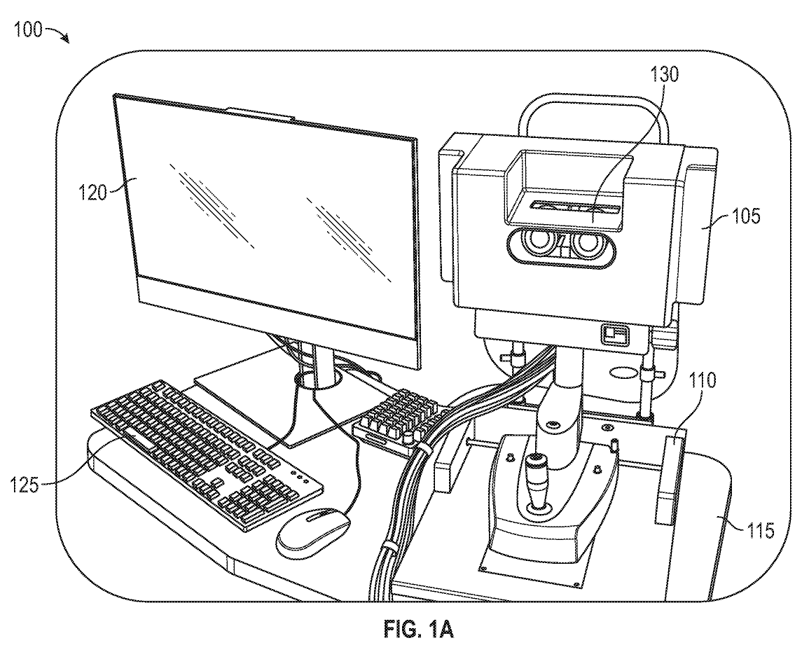

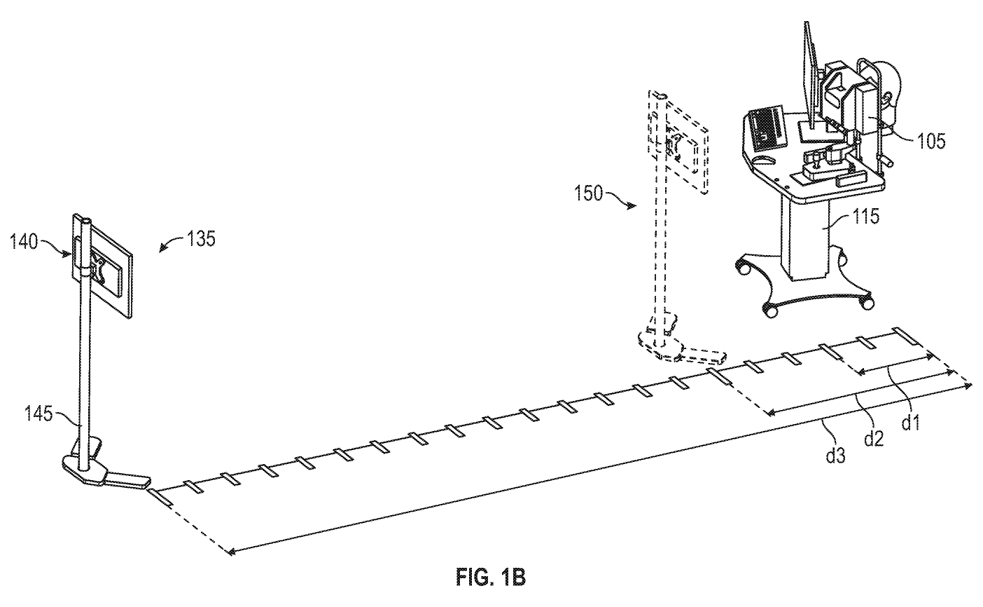

FIG. 1A illustrates a perspective view of a portion of an example optical system 100, and FIG. 1B illustrates a distant perspective view of the optical system 100, according to one or more embodiments. The optical system 100 comprises an IOL simulator device 105 that is connected to a base 110 and disposed on a table 115. The IOL simulator device 105 is communicatively connected with a display 120 and one or more input devices 125, each of which are disposed on the table 115 beside the IOL simulator device 105. The IOL simulator device 105 includes an optical system 130 that aligns with the eye(s) of a patient during a procedure such as a preclinical evaluation procedure. The optical system 130 comprises, inter alia, a removable IOL assembly through which the patient views a target 135 (shown in FIG. 1B) during the procedure. The components of the IOL simulator device 105 are discussed in greater detail below.

In FIG. 1A, a first side of the IOL simulator device 105 is depicted that generally faces toward a medical provider (e.g., a technician, clinician, or a surgeon), and faces away from the patient during the preclinical evaluation procedure. Typically, the medical provider may be seated on a first side of the table 115 facing the display 120 and the one or more input devices 125, and the patient may be seated on a second, opposing side of the table 115. The head of the patient is positioned relative to the IOL simulator device 105 to allow the patient to view through the IOL simulator device 105 to view the target 135. The IOL simulator device 105 is an upright device and may include a headrest for positioning a forehead of the patient and/or a chin rest for positioning a chin of the patient. The headrest, the chin rest, and/or the optical system 130 may be adjusted to improve the alignment and/or the viewing distance (or eye relief) of the optical system 130 with the eye(s) of the patient. Alternate implementations of the IOL simulator device 105 are also contemplated, such a headset worn by the patient during the procedure.

During a preclinical evaluation procedure using the IOL simulator device 105, one or more IOL assemblies can be easily exchanged by the medical provider (e.g., removed and/or inserted into the IOL simulator device 105 to reconfigure the characteristics of the optical system 130). In this way, the medical provider may readily assess the effects of different IOLs and/or combinations of IOLs on various aspects of the patient’s vision, which may include replicating environmental conditions such as photopic vision (bright light conditions), mesopic vision (twilight conditions), scotopic vision (low light conditions), and so forth. Beneficially, the patient may evaluate different types of IOLs and/or combinations of IOLs during the preclinical evaluation procedure, which improves the patient’s understanding of the visual effects that are achievable using the IOLs. As a result, the medical provider and the patient are able to make more informed decisions, and the likelihood of patient satisfaction with the selected IOL(s) is increased.

The target 135 includes reference information such as text, symbols, graphics, images, etc. that allow aspects of the patient’s vision to be measured or otherwise characterized using the IOL simulator device 105. For example, the reference information may include standardized information such as visual acuity charts (e.g., Snellen chart, LogMAR chart, dancing C), and may include information that tests other aspects of the patient’s vision such as contrast sensitivity, depth perception, color perception, gaze direction, pupil response, and so forth.

In some embodiments, the target 135 has a passive implementation in which the reference information is statically displayed (e.g., a printed eye chart or reference image). In other embodiments, the target 135 has an active implementation using one or more electronic devices. For example, the target 135 may include a display 140 that is controlled by the IOL simulator device 105 to display the reference information for assessing the patient’s vision.

In some embodiments, the target 135 is movable relative to the IOL simulator device 105 to allow the patient’s vision to be assessed at different distances from the display 140. As shown, the display 140 is connected to a support 145 that can be positioned at different distances between a minimum distance d₁ and a maximum distance d₃, such as a position 150 at a distance d₂ (shown in outline). For example, the support 145 may include wheels or casters allowing the support 145 to be rolled, or the entire assembly of the display 140 and the support 145 may be lifted and repositioned. In other embodiments, the target 135 may be statically positioned.

In some embodiments, the reference information of the target 135 and/or display parameters of the display 140 may be selected based on the type of procedure (e.g., which aspects of the patient’s vision are being assessed), the distance to the display 140, and so forth. In some embodiments, the medical provider uses the input device(s) 125 to provide the inputs to the IOL simulator device 105. For example, the medical provider specifies that the procedure is a visual acuity assessment and that the distance to the display 140 is four (4) meters, and the IOL simulator device 105 selects an Early Treatment Diabetic Retinopathy Study (ETDRS) chart and adjusts the size and/or spacing of the letters of the ETDRS chart on the display 140 corresponding to the distance to the display 140.

In some embodiments, the IOL simulator device 105 receives information that identifies the IOL assembly or assemblies installed in the IOL simulator device 105, and the selection of the reference information and/or the display parameters is further based on the identification of the IOL assembly or assemblies. The identification information may be provided by user input, by visual indicators of the IOL assembly or assemblies, or by wired or wireless communication with circuitry included in the IOL assembly or assemblies.

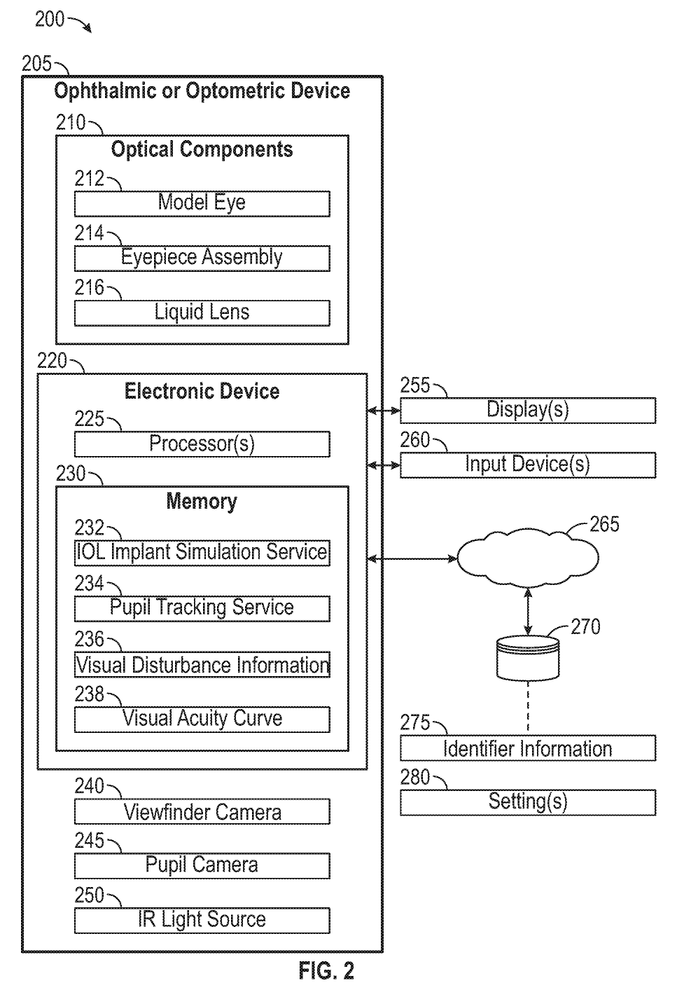

FIG. 2 illustrates a block diagram 200 of the example optical system 100 of FIGS. 1A and 1B, according to one or more embodiments. The optical system 100 comprises an ophthalmic or optometric device 205, which in some embodiments is the IOL simulator device 105 of FIGS. 1A and 1B. Other implementations of the ophthalmic or optometric device 205 are also contemplated, providing functionality in addition to IOL simulation.

The ophthalmic or optometric device 205 comprises a plurality of optical components 210, which in some embodiments includes the optical system 130 of FIG. 1A. The plurality of optical components 210 comprises a succession of optical components, such as one or more sensors, detectors, light sources, lenses, mirrors, beamsplitters, beam combiners, projection systems, reflecting prisms, filters and thin films, fiber optics, and/or the like, for performing one or more diagnostic functions of the ophthalmic or optometric device 205.

The plurality of optical components 210 is formed as different stages: a model eye 212, an eyepiece assembly 214 that is disposed proximally to the model eye 212, and in some embodiments, a liquid lens 216 that is disposed distally to the model eye 212. Note that, as described herein, the term “proximal” refers to a relative position, end, or portion of a component that is closer to a patient’s body during use thereof. On the other hand, the term “distal” refers to the relative position, end, or portion of the component that is distanced further from the patient’s body. The plurality of optical components 210 are coupled to a support structure of the ophthalmic or optometric device 205 that provides the desired arrangement of the plurality of optical components 210. A monocular configuration of the ophthalmic or optometric device 205 is described for simplicity, having a single model eye 212, eyepiece assembly 214, and liquid lens 216; however, in other embodiments, the ophthalmic or optometric device 205 has a binocular configuration with two model eyes 212, two eyepiece assemblies 214, and optionally two liquid lenses 216.

The model eye 212 comprises several components that replicate the functionality of the human eye. In some embodiments, the model eye 212 comprises an artificial cornea that is coupled to the support structure and disposed along an optical axis of the optical system 130. The artificial cornea is typically a lens having characteristics similar to a human cornea, such as a convex distal surface, a concave proximal surface, and a similar refractive index (e.g., about 1.376).

The model eye 212 further comprises an IOL assembly that is removably coupled to the support structure. For example, the IOL assembly may be inserted into a corresponding slot formed in the model eye 212, attached to an exterior surface, and so forth. In a coupled configuration, the IOL assembly is disposed proximally to the artificial cornea and is aligned with the optical axis. In some embodiments, the IOL assembly comprises an IOL disposed within a wet cell, and in some embodiments further comprises an aperture plate disposed distally to the IOL. The IOL may have any suitable implementation and provide any suitable optical characteristics.

The eyepiece assembly 214 comprises one or more lenses that are aligned with the optical axis and that magnify the image transmitted through the model eye 212 for viewing by the patient. In some embodiments, the eyepiece assembly comprises a first lens (e.g., a field lens) that is disposed distally to a second lens (e.g., an eye lens). The characteristics and disposition of the first lens and second lens may provide any suitable magnification. In some embodiments, the eyepiece assembly 214 further comprises a beamsplitter disposed proximally to the first lens (that is, disposed between the first lens and the second lens). A first surface of the beamsplitter transmits a first portion of the image transmitted through the model eye 212, and reflects a second portion of the image transmitted through the model eye 212. In some embodiments, the beamsplitter reflects the second portion of the image toward a viewfinder camera 240 of the ophthalmic or optometric device 205. In some embodiments, the optical components 210 comprise one or more additional optical components such as mirrors that redirect the reflected light from the beamsplitter.

The liquid lens 216 is aligned with the optical axis and typically comprises two immiscible liquids in a chamber: a first, conductive liquid such as water or salt solution, and a second, non-conductive liquid such as silicone oil. The first liquid and the second liquid have different refractive indices. Using different methods such as electrowetting, shape-changing polymers, or acousto-optical tuning, a control signal transmitted by a controller causes changes to a radius of curvature of the interface of the first liquid and the second liquid, altering the focal length of the liquid lens 216. The liquid lens 216 may be coupled directly or indirectly to the model eye 212 using any suitable techniques, such as being removably attached to an exterior surface or to a shared support structure.

The ophthalmic or optometric device 205 further comprises an electronic device 220 that is communicatively connected via a network 265 to a database 270. As used herein, an “electronic device” generally refers to any device having electronic circuitry that provides a processing or computing capability, and that implements logic and/or executes program code to perform various operations that collectively define the functionality of the electronic device. The functionality of the electronic device includes a communicative capability with one or more other electronic devices, e.g., when connected to a same network. An electronic device may be implemented with any suitable form factor, whether relatively static in nature (e.g., mainframe, computer terminal, server, kiosk, workstation) or mobile (e.g., laptop computer, tablet, handheld, smart phone, wearable device). The communicative capability between electronic devices may be achieved using any suitable techniques, such as conductive cabling, wireless transmission, optical transmission, and so forth.

The network 265 may have any suitable implementation, such as one or more wide area networks (WANs), one or more local access networks (LANs), or combinations thereof. The network 265 comprises infrastructure for communicative capability, such as conductive cabling, wireless transmission, optical transmission, and so forth. The network 265 may further comprise one or more electronic devices providing network functionality and/or services to the network 265, such as routers, firewalls, switches, gateway computers, edge servers, and so forth.

As shown, the electronic device 220 comprises one or more processors 225 and a memory 230. The one or more processors 225 include any electronic circuitry, including, but not limited to one or a combination of microprocessors, microcontrollers, application-specific integrated circuits (ASIC), application-specific instruction set processors (ASIP), and/or state machines, that is communicatively coupled to the memory and controls the operation of the system. In some embodiments, the electronic circuitry is configured to perform any of the functions described herein. Further, the one or more processors 225 are not limited to a single processing device and may encompass multiple processing devices.

The one or more processors 225 may include other hardware that operates software to control and process information. In some embodiments, the one or more processors 225 execute software stored in the memory 230 to perform any of the functions described herein. The one or more processors 225 control the operation and administration of the electronic device 220 by processing information (e.g., information received from input devices and/or communicatively coupled electronic devices).

The memory 230 may store, either permanently or temporarily, data, operational software, or other information for the one or more processors 225. The memory 230 may include any one or a combination of volatile or non-volatile local or remote devices suitable for storing information. For example, the memory 230 may include random-access memory (RAM), read-only memory (ROM), magnetic storage devices, optical storage devices, or any other suitable information storage device or a combination of these devices. The software represents any suitable set of instructions, logic, or code embodied in a computer-readable storage medium. For example, the software may be embodied in the memory 230, a disk, a CD, or a flash drive. In particular embodiments, the software may include an application executable by the one or more processors 225 to perform one or more of the functions described herein.

The memory 230 comprises an IOL implant simulation service 232 that in some embodiments operates as a controller of the ophthalmic or optometric device 205. In combination with one or more displays 255 (e.g., including the displays 120, 140 of FIGS. 1A and 1B, respectively) and one or more input devices 260 (e.g., including the one or more input devices 125 of FIG. 1A) that are communicatively connected to the electronic device 220, the IOL implant simulation service 232 operates to acquire data during a procedure to generate one or more visual acuity curves 238 for the patient corresponding to the IOL assembly (or assemblies).

In some embodiments, generating the one or more visual acuity curves 238 comprises one or more of the following: receiving identification information or determining characteristics of the IOL assembly or IOL assemblies; receiving indications of the type of procedure and/or display parameters; determining a distance to the display 140; and generating reference information for the display 140 based on the IOL assembly (or IOL assemblies), the type of procedure, the display parameters, and/or the distance.

In some embodiments, the IOL implant simulation service 232 uses the identification information of the IOL assembly or IOL assemblies to access the identifier information 275. The identifier information 275 comprises a plurality of model numbers and/or unique identifiers for the IOL assemblies, and one or more values of setting(s) 280 for the ophthalmic or optometric device 205, corresponding to the model numbers and/or unique identifiers. The identifier information 275 may have any suitable formatting, such as structured data or semi-structured data. For example, each unique identifier may have a record (or entry) including a plurality of fields for the setting(s) 280, and values for some or all of the plurality of fields. For example, the setting(s) 280 may include predefined focus points and/or alignments for components of the ophthalmic or optometric device 205, such that the IOL implant simulation service 232, responsive to identifying the IOL assembly or IOL assemblies, transmits control signals to implement the focus points and/or alignments.

In some embodiments, generating the one or more visual acuity curves 238 further comprises driving the display 140 to display the reference information, and/or receiving inputs indicating the patient’s responses to particular text, symbols, graphics, images, etc. of the reference information. The inputs indicating the patient’s responses may be provided by the patient or by the medical provider. For example, the patient may press keys of a keyboard corresponding to letters displayed on the display 140. In some embodiments, generating the one or more visual acuity curves 238 further comprises controlling a defocus level of the liquid lens 216.

In some embodiments, the one or more visual acuity curves 238 comprises a baseline visual acuity curve for the patient, which typically corresponds to a predefined distance of the target 135 (e.g., disposing the display 140 at four meters). The one or more visual acuity curves 238 may further comprise visual acuity curves corresponding to different IOL assemblies, different distances of the target 135, different levels of defocus, and so forth.

The memory 230 may further store visual disturbance information 236 indicating the occurrence of visual disturbances, such as halos, starbursts, and glare, that are experienced by the patient (that is, occur in the patient’s field of view) while viewing the target 135 through the ophthalmic or optometric device 205. In some embodiments, the IOL implant simulation service 232 receives inputs from the patient indicating the visual disturbances. For example, targets may be dynamically displayed using the display 140, and the patient provides input verbally or through an input device to indicate what he or she is observing. The IOL implant simulation service 232 may further capture images corresponding to the patient’s field of view using the viewfinder camera 240, and correlate the inputs to these images.

In some embodiments, the ophthalmic or optometric device 205 comprises the viewfinder camera 240 that images the field of view of the patient while viewing the target 135 through the ophthalmic or optometric device 205. In some embodiments, the beamsplitter of the eyepiece assembly 214 reflects the second portion of the image transmitted through the model eye 212 toward the viewfinder camera 240.

The IOL implant simulation service 232 may receive the images captured by the viewfinder camera 240, and store the images in the memory 230 and/or display the images to the medical provider using the display 140. The images captured by the viewfinder camera 240 may be used for a number of purposes, such as confirming optical alignment and system configuration, confirming the mounting and alignment of the IOL, confirming the general appearance of the displayed targets or information is correct, controlling and verifying that binocular pointing and vergence alignment is correct (e.g., confirming each eye is pointing at the same spot at each target distance), and showing the medical provider what the patient should be seeing.

In some embodiments, the memory 230 further comprises a pupil tracking service 234 that determines pupil alignment relative to a reference, such as an optical axis of the ophthalmic or optometric device 205. The ophthalmic or optometric device 205 comprises a pupil camera 245 that images the pupil of the patient while viewing the target 135 through the ophthalmic or optometric device 205. In some embodiments, the beamsplitter of the eyepiece assembly 214 includes a second surface that reflects an image of the pupil toward the pupil camera 245.

The pupil tracking service 234 may use image processing techniques such as object detection and/or image segmentation algorithms to define the pupil of the patient’s eye within the images and to track the pupil during the procedure. In some embodiments, the pupil camera 245 comprises an infrared (IR) camera, and the ophthalmic or optometric device 205 further comprises an IR light source 250 that is oriented toward the eye of the patient, such that the light received by the pupil camera 245 includes IR light reflected from the eye.

In some embodiments, the pupil tracking service 234 determines whether the pupil alignment is within a predefined range of the reference (e.g., the optical axis). While the pupil alignment is outside the predefined range (e.g., in a non-aligned configuration), the IOL implant simulation service 232 may invalidate or otherwise disregard the inputs indicating the patient’s responses to the text, symbols, graphics, images, etc. of the displayed reference information. In this way, the visual acuity curves 238 may be generated by the IOL implant simulation service 232 using inputs for which the patient’s pupil alignment has been validated. In some embodiments, the IOL implant simulation service 232 displays the images of the pupil and/or a graphical representation of the predefined range using the display 120. The IOL implant simulation service 232 may alert the patient and/or the medical provider when the pupil alignment is outside the predefined range, e.g., visually or audibly reminding the patient to look forward or remain still during the procedure. The pupil tracking service 234 may provide additional functionality, such as gaze tracking when displaying targets in different positions during the procedure.

### Example IOL Simulator

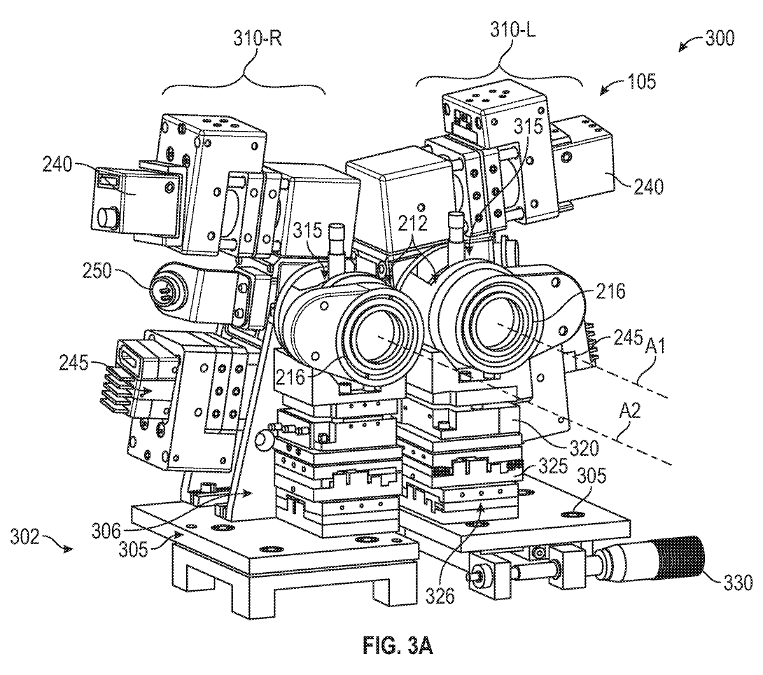

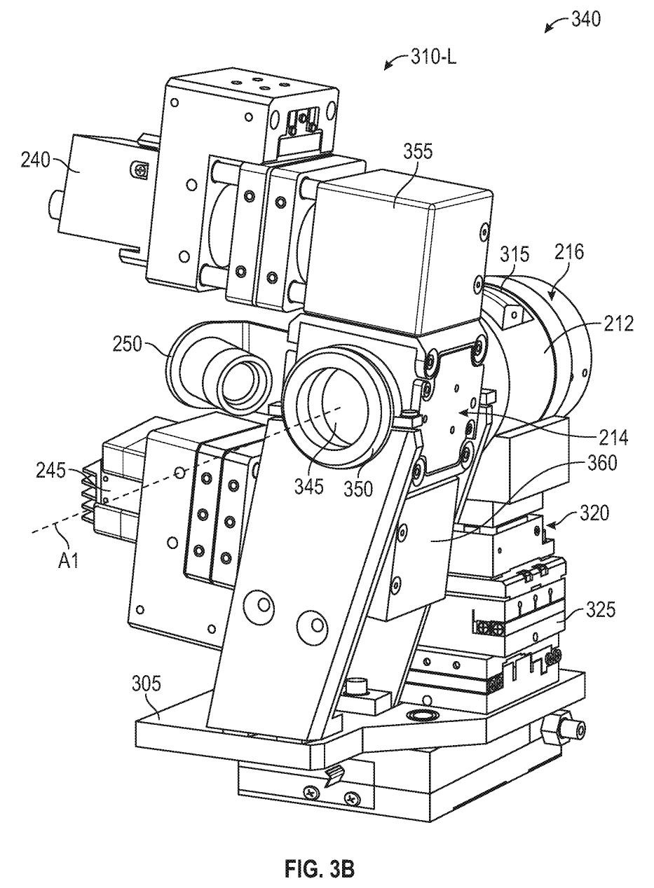

FIG. 3A illustrates a perspective view 300 of a distal end of the IOL simulator device 105, and FIG. 3B illustrates a perspective view 340 of a proximal end of a monocular subassembly 310-L of the IOL simulator device 105, according to one or more embodiments. The IOL simulator device 105 has a binocular implementation, where two monocular subassemblies 310-L, 310-R are connected to a common support structure 302. Each of the monocular subassemblies 310-L, 310-R may have similar components and functions, except as otherwise specified. The IOL simulator device 105 may be substantially symmetrical, such that the monocular subassemblies 310-L, 310-R may substantially mirror each other about a central plane of the IOL simulator device 105.

The support structure 302 includes one or more horizonal platforms 305 with one or more vertical projections 306 extending therefrom. Various components of the IOL simulator device 105 may be removably connected to the one or more horizontal platforms 305 and/or the one or more vertical projections 306, e.g., using threaded fasteners.

The IOL simulator device 105 comprises, from the distal end to the proximal end along each optical axis A1, A2, the liquid lens 216, the model eye 212, and the eyepiece assembly 214. The IOL simulator device 105 further comprises, connected to a bottom surface of each model eye 212 and disposed between the model eye 212 and the horizontal platform 305, a pointing alignment 320, a motorized focus stage 325, and a vergence stage 326. The pointing alignment 320, the motorized focus stage 325, and the vergence stage 326 may be communicatively connected to the electronic device 220 and controlled by signals transmitted by the IOL implant simulation service 232. In some embodiments, the vergence stage 326 is used to control pointing at different target distances for the purpose of evaluating visual acuity at multiple distances. The IOL simulator device 105 further comprises an interpupillary distance (IPD) adjustment 330 connected to one horizontal platform 305, which in some embodiments may be manually rotated to change a distance between portions of the two monocular subassemblies 310-L, 310-R. Each model eye 212 defines an opening into which a respective IOL assembly 315 is inserted to arrange an IOL along each optical axis A1, A2. As shown, the opening extends from a top surface of the model eye 212 opposite to the bottom surface.

In some embodiments, the eyepiece assembly 214 comprises a beamsplitter that transmits a first portion of the image transmitted through the model eye 212, toward an eye cup 350 formed in an opening 345 at the proximal end of the IOL simulator device 105. The beamsplitter reflects a second portion of the image along a first optical path toward the viewfinder camera 240. As shown, the IOL simulator device 105 further comprises a reflector 355 disposed along the first optical path. The beamsplitter reflects the second portion of the image in a first (as shown, generally upward) direction, and the reflector 355 redirects the second portion of the image in a substantially orthogonal (lateral) direction toward the viewfinder camera 240.

The IOL simulator device 105 further comprises the pupil camera 245 for imaging the pupil of the patient. In some embodiments, the IOL simulator device 105 further comprises the IR light source 250 oriented toward the eye of the patient. IR light that is emitted by the IR light source 250 is reflected from the eye and received through the opening 345. A second surface of the beamsplitter reflects a portion of the received light along a second optical path toward the pupil camera 245. As shown, the IOL simulator device 105 further comprises a reflector 360 disposed along the second optical path. The beamsplitter reflects the portion of the received light in a first (as shown, generally downward) direction, and the reflector 360 redirects the portion of the received light in a substantially orthogonal (lateral) direction toward the pupil camera 245.

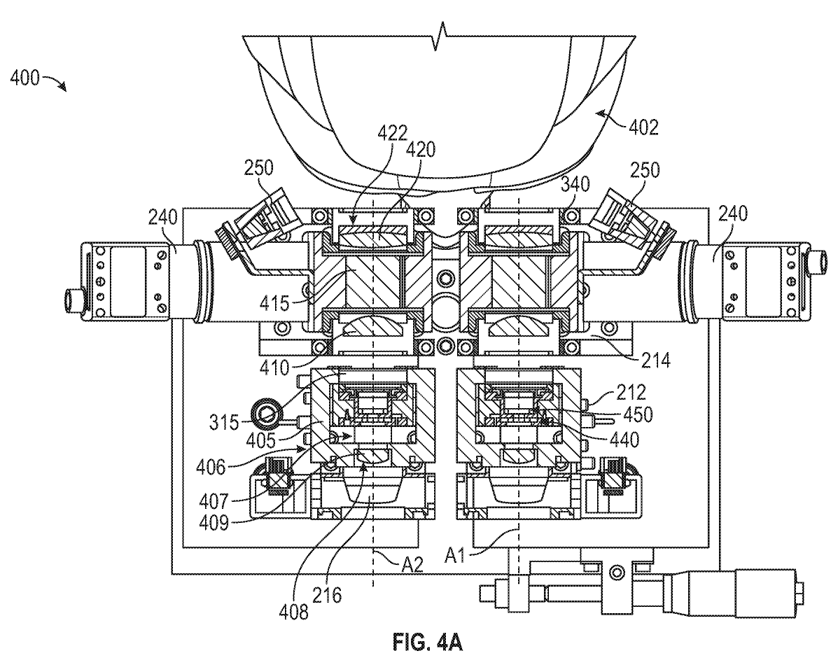

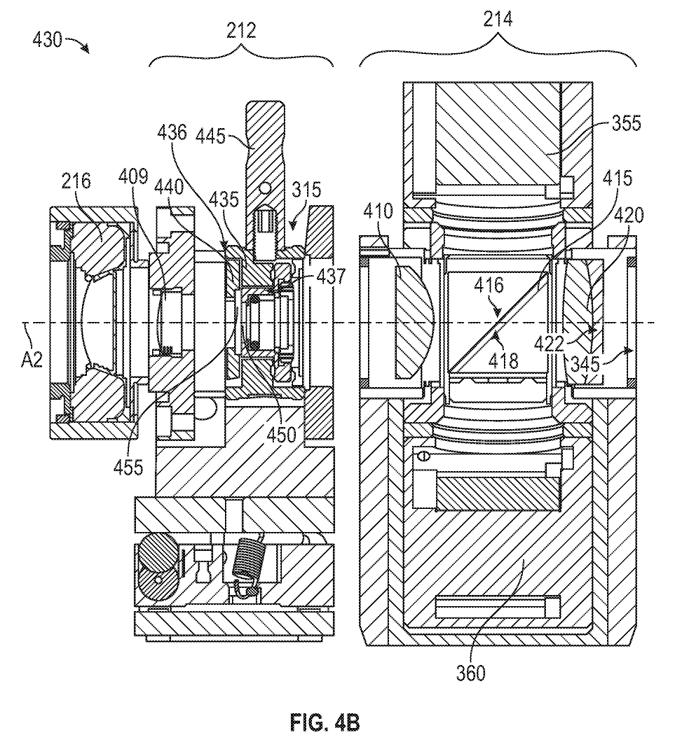

FIG. 4A illustrates a top cross-section view 400 of the example IOL simulator device 105 of FIG. 3A with a patient 402, and FIG. 4B illustrates a side cross-section view 430 of the example IOL simulator device 105 of FIG. 3A, according to one or more embodiments.

The model eye 212 comprises a body 405 defining an exterior surface 406 and an interior volume 407. The exterior surface 406 may have any suitable contouring, such as being substantially cylindrical. In some embodiments, the body 405 defines an opening extending through the exterior surface 406, such that the IOL assembly 315 may be inserted into and removed from the interior volume 407. The body 405 may define one or more alignment features and/or retention features that contact corresponding portions of the IOL assembly 315 to align the IOL assembly 315 with the optical axis A1, A2 when the IOL assembly 315 is in a coupled configuration.

The IOL assembly 315 comprises a body 435 defining an exterior surface 436, a handle 445 extending from the exterior surface 436, and an interior volume 437. The IOL assembly 315 further comprises an IOL wet cell 450 disposed within the interior volume 437, and the IOL 455 is disposed within the IOL wet cell 450. One or more components of the IOL wet cell 450 may retain the IOL 455 with a desired positioning. The handle 445 allows the IOL assembly 315 to be more easily manipulated by a medical provider when inserting or removing the IOL assembly 315 from the body 405 of the model eye 212. In some embodiments, the IOL assembly 315 may be referred to as an IOL coin, the body 435 having the shape of a circular disc, that is interchangeable within the model eye 212.

In some embodiments, the IOL assembly 315 further comprises a removable aperture plate 440 that connects to the body 435 using any suitable techniques. For example, the aperture plate 440 may be disposed in a recess formed from a distal surface of the body 435, and retained in the recess using magnetized features or threaded fasteners. The aperture of the aperture plate 440 is aligned with the optical axis A1, A2 when in the retained configuration. The aperture plate 440 allows different aspects of the patient’s vision to be assessed, such as photopic vision, mesopic vision, distance vision, near vision, and so forth. In some embodiments, the aperture plate 440 includes a glass window with a deposited chrome aperture, where multiple diameters may be available for the different vision test scenarios including photopic (small pupil, bright light) and mesopic (large pupil, low light) vision testing. Other embodiments of the aperture plate 440 may simulate non-circular iris disease states including iris coloboma, iridodialysis, and so forth.

An artificial cornea 409 is connected to the body 405 and disposed along the optical axis A1, A2. In some embodiments, the artificial cornea 409 is disposed in a recess 408 extending from a distal surface of the body 405. The artificial cornea 409 typically simulates human cornea optical properties for the purposes of IOL testing. Embodiments of the artificial cornea 409 may have different power levels, spherical aberration (SA), and/or chromatic aberration profiles. The artificial cornea 409 may further simulate other optical properties including astigmatism, high-order aberrations, or simulated corneal conditions (e.g., post-LASIK cornea). In some embodiments, the artificial cornea 409 is spaced apart from the IOL assembly 315 and separated by free space.

In some embodiments, the eyepiece assembly 214 is spaced apart from the model eye 212 and separated by free space. The eyepiece assembly 214 comprises a first lens 410 (as shown, a plano-convex lens), a beamsplitter 415 disposed proximally to the first lens 410, and a second lens 420 (as shown, a biconvex lens) disposed proximally to the beamsplitter 415. In some embodiments, the eyepiece assembly 214 further comprises a third lens 422 (as shown, a concavo-convex lens) disposed proximally to the second lens 420. The beamsplitter 415 defines a first (distal) surface 416 that reflects a portion of light incident on the model eye 212 toward the reflector 355, and a second (proximal) surface 418 that reflects a portion of the received light through the opening 345 toward the reflector 360.

### Example Model Eye

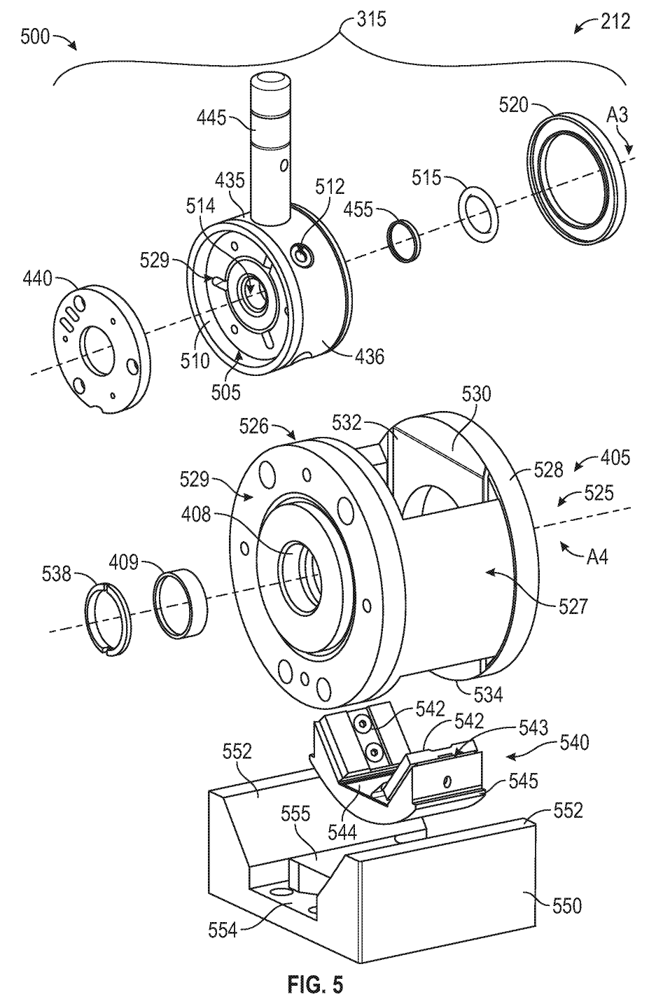

FIG. 5 illustrates an exploded view 500 of the model eye 212 with the removable IOL assembly 315, according to one or more embodiments. The body 435 of the IOL assembly 315 defines a distal surface 505 and a recess 510 formed from the distal surface 505. The recess 510 is dimensioned to receive the aperture plate 440 and in some cases, to retain the aperture plate 440 to the body 435. The aperture plate 440 is aligned with an optical axis A3 of the removable IOL assembly 315.

The body 435 further defines a wet cell window 514, which, along with the IOL 455 and an IOL retaining ring 515, forms the IOL wet cell 450. The removable IOL assembly 315 further comprises a wet cell back window retaining ring 520 that attaches to the body 435 and defines a proximal surface of the IOL assembly 315. The body 435 also defines openings for one or more IOL alignment adjustment mechanisms 512, which in some cases are orthogonally-arranged screw-springs that can adjust the position of the IOL 455 to align with the optical axis of the removable IOL assembly 315.

The body 405 of the model eye 212 comprises a base 525 having a generally cylindrical shape. The base 525 comprises a first plate section 526 and a section plate section 528, and an annular section 527 disposed therebetween. The first plate section 526 and the second plate section 528 may have a first material composition such as aluminum or stainless steel, and the annular section 527 may have a second material composition such as polycarbonate (PC), acrylonitrile butadiene styrene (ABS), or composites thereof.

The annular section 527 defines an interior volume 530, a first circumferential opening 532, and a second circumferential opening 534. In some embodiments, the first circumferential opening 532 and the second circumferential opening 534 are disposed opposite each other. As shown, the first circumferential opening 532 is disposed near a top of the base 525, and the second circumferential opening 534 is disposed near a bottom of the base 525.

The first plate section 526 defines a distal end opening in which the recess 408 is defined, and the second plate section 528 defines a proximal end opening. The recess 408 extends into the first plate section 526 from a distal surface 529 of the base 525. The artificial cornea 409 is received into the recess 408 and is aligned with an optical axis A4 of the base 525. The artificial cornea 409 is retained in the recess 408 using a retaining ring 538 disposed distally to the artificial cornea 409.

The IOL assembly 315 is received into the interior volume 530 of the annular section 527 through the first circumferential opening 532. In some embodiments, a first alignment component 540 is received into the second circumferential opening 534 and connected to the base 525. For example, features of the first alignment component 540 may seat against corresponding features of the annular section 527 to place the first alignment component 540 into a coupled configuration. The first alignment component 540 may be attached to the annular section 527 in the coupled configuration using, e.g., threaded fasteners.

In some embodiments, the first alignment component 540 has an exterior surface 545 that is contoured to form a substantially continuous external surface with the annular section 527 when in the coupled configuration. The first alignment component 540 further defines one or more alignment features that extend into the interior volume 530 when in the coupled configuration. As shown, the alignment features include opposing angled surfaces 542 spaced apart from each other, and a substantially flat surface 544 disposed between the angled surfaces 542. The IOL assembly 315 when received into the interior volume 530 seats against the angled surfaces 542 and/or the substantially flat surface 544 to align the optical axis A3 of the IOL assembly 315 with the optical axis A4 of the base 525. Further, in some embodiments, adjustable shims 543 are installed beneath the angled surfaces 542, allowing micro-adjustments to ensure alignment of optical axis A3 and A4 by adjusting the shim thickness beneath the angled surfaces 542.

In the coupled configuration, the base 525 seats against a second alignment component 550 that aligns the optical axis A4 of the base 525 (and the optical axis A3 of the IOL assembly 315 when inserted) with the optical axis A1, A2 of the IOL simulator device 105. In some embodiments, the first alignment component 540 contacts an upper surface of the second alignment component 550 when in the coupled configuration. As shown, the second alignment component 550 includes opposing angled surfaces 552 spaced apart from each other, a substantially flat surface 554 disposed between the angled surfaces 552, and a substantially flat raised surface 555. The base 525 seats against the angled surfaces 552, the substantially flat surface 554, and/or the substantially flat raised surface 555 to align the optical axis A4 of the base 525 with the optical axis A1, A2 of the IOL simulator device 105. In some alternate embodiments, the first alignment component 540 and the second alignment component 550 may include complementary alignment features that align the optical axis A4 of the base 525 with the optical axis A1, A2 of the IOL simulator device 105.

### Example System Diagram of IOL Simulator

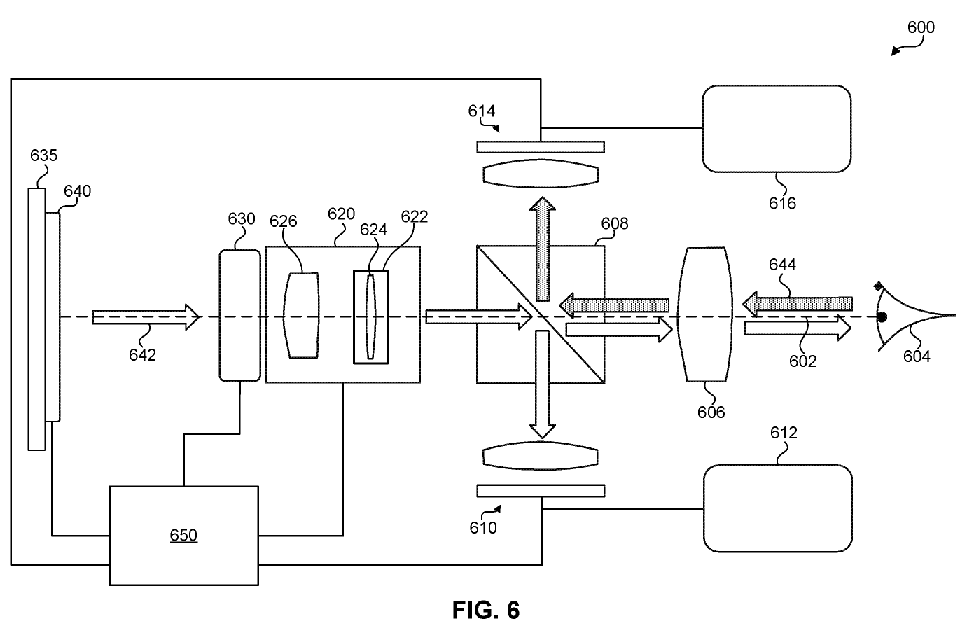

FIG. 6 illustrates a system diagram of an example IOL simulator system 600, according to an example embodiment. IOL simulator system 600 may be similar to the optical system 100 described above with reference to FIGS. 1A, 1B, and 2, and may be similar to the IOL simulator device 105 described above with reference to FIGS. 1A–5. Although IOL simulator system 600 is shown in FIG. 6 as a stand-alone apparatus and/or system, the aspects of this disclosure may be used with other apparatuses, systems, and/or methods, such as, but not limited to, elements in FIGS. 1A–5 and 7–10, e.g., optical system 100, IOL simulator device 105, ophthalmic or optometric device 205, viewfinder display 700, viewfinder display 800, pupil display 900, and/or flow diagram 1000.

IOL simulator system 600 may be configured to provide controlled testing of vision under different conditions (e.g., photopic, mesopic, scotopic) as well as different distances (e.g., using adjustable targets and displays) to simulate IOL implantation before surgery. IOL simulator system 600 may be further configured to enable virtual evaluation and comparison of different IOL designs (e.g., for each patient eye) before surgery. IOL simulator system 600 may be further configured to provide dynamic focusing capability (e.g., real time adjustments) using a liquid lens and a model eye with an artificial cornea and interchangeable IOL assemblies. IOL simulator system 600 may be further configured to evaluate performance of an IOL assembly and any visual disturbances experienced by a patient before surgery using iterative and controllable objective assessment via one or more evaluation parameters (e.g., horizontal error, vergence error, vertical error, left viewfinder focus score, left viewfinder focus position, right viewfinder focus score, right viewfinder focus position, left pupil alignment score, right pupil alignment score).

As shown in FIG. 6, IOL simulator system 600 may include an optical axis 602, an eyepiece assembly 606, a beamsplitter 608, a viewfinder camera 610, a viewfinder display 612, a pupil camera 614, a pupil display 616, a model eye 620, a liquid lens 630, a target 635, and a controller 650. Optical axis 602 may extend through IOL simulator system 600 such that eyepiece assembly 606, beamsplitter 608, model eye 620, liquid lens 630, and target 635 are aligned along optical axis 602. IOL simulator system 600 allows a patient 604 to look through eyepiece assembly 606 (e.g., monocular or binocular) and view target 635 through model eye 620, that includes an IOL 624, to assess visual quality and any visual disturbances caused by optical scattering and/or aberrations from IOL 624. Viewfinder camera 610 and pupil camera 614 are included in opposing optical paths, separated by beamsplitter 608, to allow an operator to observe what patient 604 is viewing in real time (e.g., via viewfinder display 612) as well as track and measure a pupil of patient 604 simultaneously (e.g., via pupil display 616). Liquid lens 630 on each monocular channel allows for the simulation of defocus, which can change the apparent distance of target 635 (e.g., an external scene) being observed by patient 604.

Model eye 620 may be configured to simulate a human eye having undergone an IOL implantation. As shown in FIG. 6, model eye 620 may include an IOL assembly 622 with IOL 624 and an artificial cornea 626 disposed proximal to IOL 624. In some embodiments, different IOLs may be loaded into each monocular channel such that binocular summation or monovision may be simulated and quality of vision assessed. Target 635 may be configured to present an external scene to patient 604 through model eye 620. As shown in FIG. 6, target 635 may include a target display 640 that projects visual reference information 642 along optical axis 602 toward patient 604 along a first optical path. Patient response data 644 (e.g., pupil alignment, visual disturbances) from having viewed visual reference information 642 through model eye 620 may be detected via pupil camera 614 along a second optical path.

Controller 650 may be configured to generate an assessment output (e.g., a visual acuity curve) based on visual reference information 642 and patient response data 644 to characterize visual performance associated with IOL assembly 622. Controller 650 may be further configured to iteratively control one or more components of IOL simulator system 600 to test vision under different conditions (e.g., different external scenes) as well as at different distances (e.g., adjusting defocus) to simulate IOL implantation in various environments and conditions. As shown in FIG. 6, controller 650 may be in communication with viewfinder camera 610, pupil camera 614, model eye 620 (e.g., IOL assembly 622), liquid lens 630, and target display 640. In this way, controller 650 may be configured to identify IOL assembly 622 for evaluation, select appropriate visual reference information 642 (e.g., via target display 640), select an appropriate defocus level (if needed) (e.g., via adjusting liquid lens 630), monitor viewfinder camera 610 to confirm visual reference information 642 presented to patient 604 through model eye 620 is correct, and monitor pupil camera 614 to evaluate patient response data 644 (e.g., pupil alignment).

In some embodiments, controller 650 may be configured to measure one or more evaluation parameters to assess IOL assembly 622 during a preclinical evaluation procedure. In some embodiments, for example, the one or more evaluation parameters may include a horizontal error, a vergence error, a vertical error, a left viewfinder focus score, a left viewfinder focus position, a right viewfinder focus score, a right viewfinder focus position, a left pupil alignment score, a right pupil alignment score, or a combination thereof. In some embodiments, controller 650 may be configured to generate a visual acuity curve corresponding to IOL assembly 622 based on the preclinical evaluation process. In some embodiments, controller 650 may be configured to store information indicative of one or more visual disturbances experienced by patient 604 while viewing target 635 (e.g., visual reference information 642) through model eye 620. In some embodiments, for example, the one or more visual disturbances may include glare, halos, starbursts, reduced contrast, depth of focus artifacts, or a combination thereof.

In some embodiments, controller 650 may repeat a preclinical evaluation process for a second IOL assembly, where the second IOL assembly has at least one different optical characteristic than IOL assembly 622. In some embodiments, for example, controller 650 may compare assessment outputs between IOL assembly 622 and the second IOL assembly. In some embodiments, controller 650 may be configured to introduce a plurality of defocus levels (e.g., via controlling liquid lens 630) while patient 604 views target 635 and generate a plurality of visual acuity curves corresponding to the plurality of defocus levels. In some embodiments, controller 650 may be configured to track a position of a pupil of an eye of patient 604 and generate, based on the position of the pupil, a visual acuity curve for the eye. In some embodiments, controller 650 may be configured to capture images of a pupil of an eye of patient 604 while viewing target 635 (e.g., via pupil camera 614) and validate inputs indicative of patient response data 644 based on whether the pupil is aligned within a predefined range relative to optical axis 602.

In operation, controller 650 identifies IOL assembly 622 for evaluation (e.g., via an identifier, operator input) and IOL simulator system 600 is initialized (e.g., IOL assembly 622 is aligned along optical axis 602) for a preclinical evaluation process. A patient (e.g., patient 604) is positioned to view target 635 through model eye 620 along optical axis 602. Controller 650 selects (e.g., via operator input) appropriate visual reference information 642 and projects it from target display 640. Visual reference information 642 passes through liquid lens 630, model eye 620, and beamsplitter 608, which sends a portion of the visual reference information 642 to viewfinder camera 610 for evaluation, and then through eyepiece assembly 606 where it is viewed by patient 604. At this time, controller 650 also acquires patient response data 644 (e.g., pupil alignment) via a reflected light path from patient 604 that passes back through eyepiece assembly 606 and is reflected by beamsplitter 608, which sends the patient response data 644 to pupil camera 614 for evaluation.

In some embodiments, IOL assembly 622 may be selected from one of a plurality of interchangeable IOL assemblies each providing a different optical characteristic. In some embodiments, visual reference information 642 may include optotypes, contrast patterns, defocus targets, simulated night driving scenes, or a combination thereof at a simulated distance. In some embodiments, patient response data 644 may include user input indicative of visual acuity, contrast sensitivity, subjective visual quality, or a combination thereof. In some embodiments, visual reference information 642 and an assessment output (e.g., a visual acuity curve) determined by controller 650 are based on received information identifying IOL assembly 622, a vision assessment procedure (e.g., optotypes, contrast patterns, defocus targets, simulated night driving scenes, photopic, mesopic, scotopic, or a combination thereof), or both.

### Example Displays of IOL Simulator

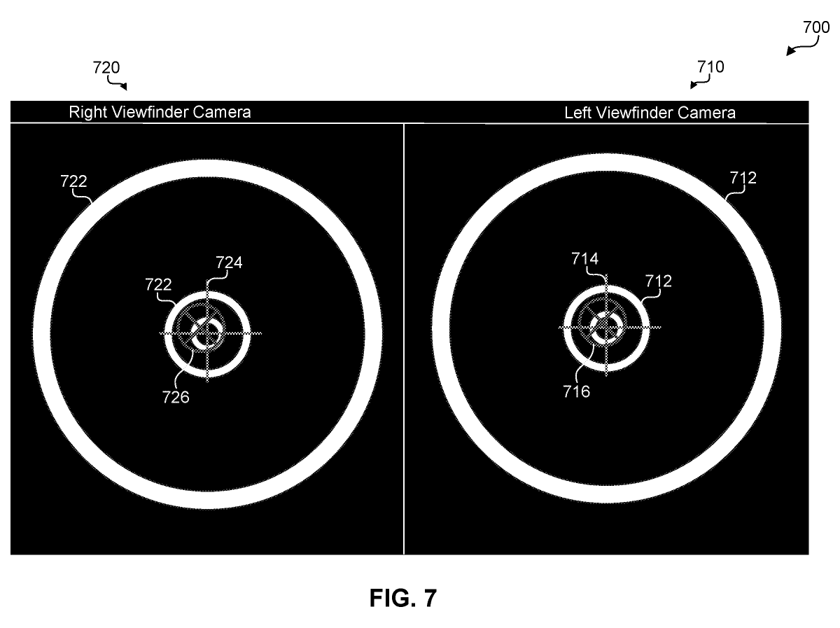

FIGS. 7–9 illustrate example displays of IOL simulator system 600, according to various example embodiments. Although viewfinder display 700, viewfinder display 800, and pupil display 900 are shown in FIGS. 7–9 as a stand-alone apparatus and/or system, the aspects of this disclosure may be used with other apparatuses, systems, and/or methods, such as, but not limited to, elements in FIGS. 1A–6 and 10, e.g., optical system 100, IOL simulator device 105, ophthalmic or optometric device 205, IOL simulator system 600, and/or flow diagram 1000.

FIG. 7 shows viewfinder display 700 for a preclinical evaluation procedure using IOL simulator system 600, according to an example embodiment. As shown in FIG. 7, viewfinder display 700 shows left viewfinder camera 710 and right viewfinder camera 720 displaying an external scene (e.g., left and right visual reference information 712, 722) for evaluation. Left viewfinder camera 710 corresponds to a left monocular channel presenting left visual reference information 712 from a target (e.g., target 635 shown in FIG. 6), and right viewfinder camera 720 corresponds to a right monocular channel presenting right visual reference information 722 from the target. As shown in FIG. 7, left viewfinder camera 710 includes left visual reference information 712 (e.g., a bulls-eye pattern), a left current position 714 (e.g., a cross-hair), and a left target position 716 (e.g., a circled-X), and right viewfinder camera 720 includes right visual reference information 722 (e.g., a bulls-eye pattern), a right current position 724 (e.g., a cross-hair), and a right target position 726 (e.g., a circled-X).

In some embodiments, left and right visual reference information 712, 722 may include optotypes, contrast patterns, defocus targets, simulated night driving scenes, or a combination thereof. For example, as shown in FIG. 7, left and right visual reference information 712, 722 includes a bulls-eye pattern (e.g., white concentric rings). In some embodiments, viewfinder display 700 may correspond to a predetermined distance and/or magnification for a preclinical evaluation procedure. In some embodiments, for example, viewfinder display 700 may correspond to near vision (e.g., object distance at 0.3 m) or a high magnification (e.g., 4x) for evaluation.

In some embodiments, viewfinder display 700 may be used to calculate one or more evaluation parameters for an objective assessment of an IOL assembly (e.g., a horizontal error, a vergence error, a vertical error, a left viewfinder focus score, a left viewfinder focus position, a right viewfinder focus score, a right viewfinder focus position, or a combination thereof). In some embodiments, for example, a position difference between left current position 714 and left target position 716 may be used to calculate a left horizontal error, a left vergence error, a left vertical error, a left viewfinder focus score, and a left viewfinder focus position. In some embodiments, for example, a position difference between right current position 724 and right target position 726 may be used to calculate a right horizontal error, a right vergence error, a right vertical error, a right viewfinder focus score, and a right viewfinder focus position. In some embodiments, for example, an average of the left and right horizontal errors may be used to determine a horizontal error for the IOL assembly. In some embodiments, for example, the left and right vergence errors may be used to determine a vergence error for the IOL assembly. In some embodiments, an average of the left and right vertical errors may be used to determine a vertical error for the IOL assembly.

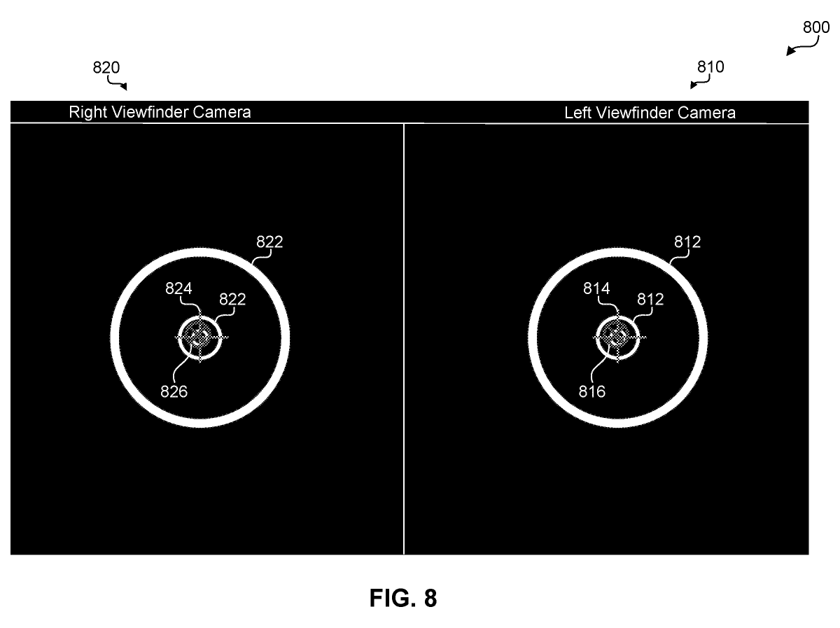

FIG. 8 shows viewfinder display 800 for a preclinical evaluation procedure using IOL simulator system 600, according to an example embodiment. Similar to FIG. 7, as shown in FIG. 8, viewfinder display 800 shows left viewfinder camera 810 and right viewfinder camera 820 displaying an external scene (e.g., left and right visual reference information 812, 822) for evaluation. Left viewfinder camera 810 corresponds to a left monocular channel presenting left visual reference information 812 from a target (e.g., target 635 shown in FIG. 6), and right viewfinder camera 820 corresponds to a right monocular channel presenting right visual reference information 822 from the target. As shown in FIG. 8, left viewfinder camera 810 includes left visual reference information 812 (e.g., a distant bulls-eye pattern), a left current position 814 (e.g., a cross-hair), and a left target position 816 (e.g., a circled-X), and right viewfinder camera 820 includes right visual reference information 822 (e.g., a distant bulls-eye pattern), a right current position 824 (e.g., a cross-hair), and a right target position 826 (e.g., a circled-X).

In some embodiments, left and right visual reference information 812, 822 may include optotypes, contrast patterns, defocus targets, simulated night driving scenes, or a combination thereof. For example, as shown in FIG. 8, left and right visual reference information 812, 822 includes a distant bulls-eye pattern (e.g., distant white concentric rings). In some embodiments, viewfinder display 800 may correspond to a predetermined distance and/or magnification for a preclinical evaluation procedure. In some embodiments, for example, viewfinder display 800 may correspond to distance vision (e.g., object distance at 6 m) or no magnification (e.g., 1x) for evaluation. In some embodiments, viewfinder display 800 may be used to calculate one or more evaluation parameters for an objective assessment of an IOL assembly (e.g., a horizontal error, a vergence error, a vertical error, a left viewfinder focus score, a left viewfinder focus position, a right viewfinder focus score, a right viewfinder focus position, or a combination thereof).

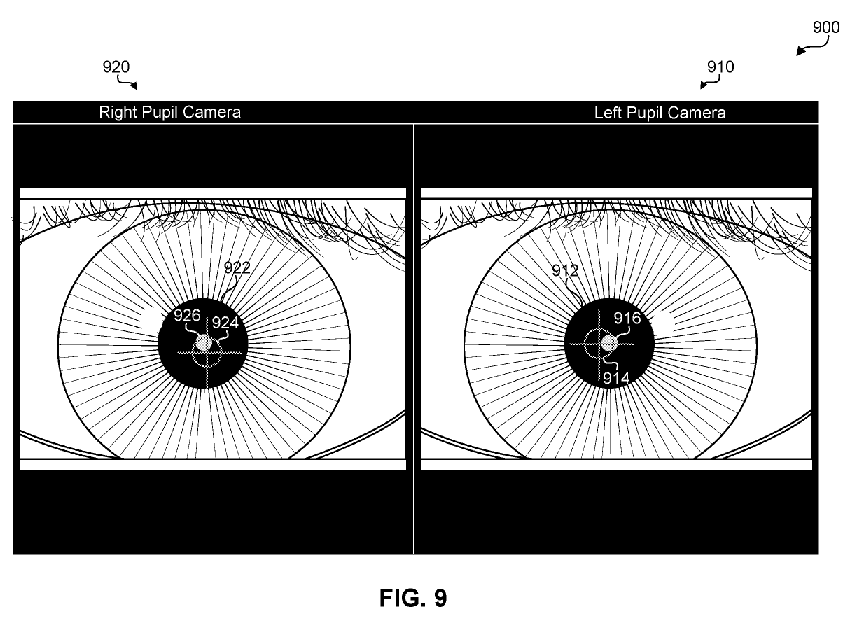

FIG. 9 shows pupil display 900 for a preclinical evaluation procedure using IOL simulator system 600, according to an example embodiment. As shown in FIG. 9, pupil display 900 shows left pupil camera 910 and right pupil camera 920 displaying a patient’s left and right eyes with left and right pupils 912, 922 for evaluation. Left pupil camera 910 corresponds to a left monocular channel imaging the patient’s left pupil 912, and right pupil camera 920 corresponds to a right monocular channel imaging the patient’s right pupil 922. As shown in FIG. 9, left pupil camera 910 includes the patient’s left pupil 912, a left optical axis alignment marker 914 (e.g., a cross-hair), and a left pupil indicator 916 (e.g., a colored dot), and right pupil camera 920 includes the patient’s right pupil 922, a right optical axis alignment marker 924 (e.g., a cross-hair), and a right pupil indicator 926 (e.g., a colored dot). In some embodiments, a position of left pupil indicator 916 and right pupil indicator 926 may be used to calculate the patient’s IPD. In some embodiments, a position difference between left optical axis alignment marker 914 and left pupil indicator 916 may be used to calculate a left pupil alignment score. In some embodiments, a position difference between right optical axis alignment marker 924 and right pupil indicator 926 may be used to calculate a right pupil alignment score.

In some embodiments, the patient’s IPD, the left pupil alignment score, and/or the right pupil alignment score may be used in combination with measurements from viewfinder display (e.g., viewfinder display 700, 800) for the same target to calculate one or more evaluation parameters for an objective assessment of an IOL assembly (e.g., a horizontal error, a vergence error, a vertical error, a left viewfinder focus score, a left viewfinder focus position, a right viewfinder focus score, a right viewfinder focus position, or a combination thereof).

### Example Flow Diagram

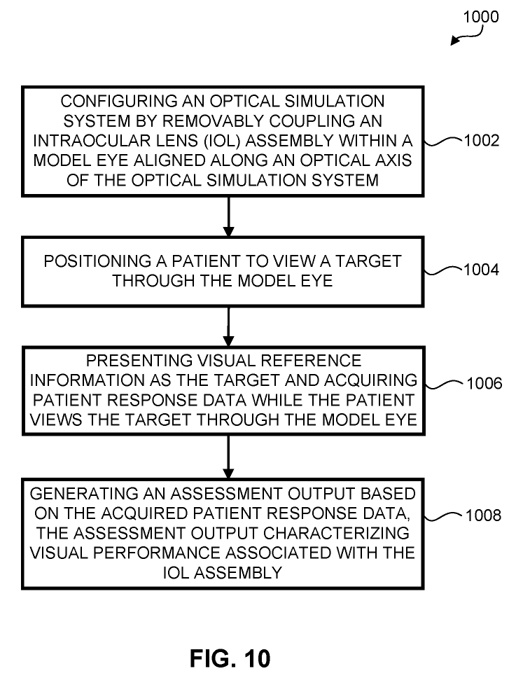

FIG. 10 illustrates flow diagram 1000 according to an example embodiment. For example, flow diagram 1000 may be for optical system 100 with IOL simulator device 105 shown in FIGS. 1A and 1B. For example, flow diagram 1000 may be for IOL simulator system 600 shown in FIG. 6. Flow diagram 1000 may be configured to provide controlled testing of vision under different conditions (e.g., photopic, mesopic, scotopic) as well as different distances (e.g., using adjustable targets and displays) to simulate IOL implantation before surgery. Flow diagram 1000 may be further configured to enable virtual evaluation and comparison of different IOL designs (e.g., for each patient eye) before surgery. Flow diagram 1000 may be further configured to utilize objective assessment of IOL assembly performance and any visual disturbances to simulate IOL implantation before surgery.

It is to be appreciated that not all operations in FIG. 10 are needed to perform the disclosure provided herein. Further, some of the operations may be performed simultaneously, sequentially, and/or in a different order than shown in FIG. 10. Flow diagram 1000 shall be described with reference to FIGS. 1A–9. However, flow diagram 1000 is not limited to those example embodiments. Although flow diagram 1000 is shown in FIG. 10 as a stand-alone method, the embodiments of this disclosure can be used with other apparatuses, systems, and/or methods, such as, but not limited to, elements in FIGS. 1A–9, e.g., optical system 100, IOL simulator device 105, ophthalmic or optometric device 205, IOL simulator system 600, viewfinder display 700, viewfinder display 800, and/or pupil display 900. In some embodiments, flow diagram 1000 may be implemented by one or more models or algorithms (e.g., an optimization algorithm) run on one or more processors and/or computing devices based on one or more instructions stored in one or more memories (e.g., electronic device 220, controller 650).

In operation 1002, as shown in the example of FIGS. 1A–9, an optical simulation system (e.g., optical system 100, IOL simulator system 600) is configured by removably coupling an IOL assembly (e.g., IOL assembly 315, IOL assembly 622) within a model eye (e.g., model eye 212, model eye 620) aligned along an optical axis of the optical simulation system. In some embodiments, removably coupling the IOL assembly may include selecting one of a plurality of interchangeable IOL assemblies each providing a different optical characteristic. In some embodiments, removably coupling the IOL assembly may include selecting an aperture plate associated with a simulated lighting condition. In some embodiments, flow diagram 1000 may further include simulating at least one of photopic vision, mesopic vision, or scotopic vision while presenting the target.

In operation 1004, as shown in the example of FIGS. 1A–9, a patient (e.g., patient 402, patient 604) is positioned to view a target (e.g., target 135, target 635) through the model eye. In some embodiments, positioning the patient may include positioning the patent relative to an eyepiece assembly (e.g., eyepiece assembly 214, eyepiece assembly 606) such that at least one eye of the patient is aligned with the optical axis to view the target through the model eye.

In operation 1006, as shown in the example of FIGS. 1A–9, visual reference information (e.g., display 140, visual reference information 642) is presented as the target and patient response data (e.g., pupil tracking service 234, visual disturbance information 236, patient response data 644) is acquired (e.g., via pupil camera 614) while the patient view the target through the model eye. In some embodiments, presenting the visual reference information may include displaying optotypes, contrast patterns, defocus targets (e.g., bulls-eye), simulated night driving scenes, or a combination thereof at a simulated distance. In some embodiments, acquiring patient response data may include receiving user input indicative of visual acuity, contrast sensitivity, subjective visual quality, or a combination thereof. In some embodiments, presenting the visual reference information may include presenting the target at a plurality of distances from the model eye. In some embodiments, the visual reference information may be scaled or selected based on a distance at which the target is presented.

In some embodiments, flow diagram 1000 may further include tracking (e.g., via controller 650) a position of a pupil of an eye of the patient. In some embodiments, for example, flow diagram 1000 may further include generating, based on the position of the pupil, a visual acuity curve for the eye. In some embodiments, flow diagram 1000 may further include capturing images of a pupil of an eye of the patient while the patient views the target and validating inputs indicative of patient responses based on whether the pupil is aligned within a predefined range relative to the optical axis. In some embodiments, flow diagram 1000 may further include disregarding inputs indicative of patient responses acquired while the pupil is outside the predefined range. In some embodiments, flow diagram 1000 may further include providing an alert to the patient or an operator when the pupil is determined to be outside the predefined range.

In some embodiments, flow diagram 1000 may further include receiving (e.g., via controller 650) information indicative of at least one of (i) an identification of the selected IOL assembly, (ii) a type of vision assessment procedure to be performed, or (iii) a target distance or a display parameter associated with the target. In some embodiments, when receiving information indicative of an identification of the IOL assembly, flow diagram 1000 may further include detecting an identifier associated with the selected IOL assembly and applying, based on the identifier, one or more settings for a preclinical evaluation procedure. In some embodiments, flow diagram 1000 may further include receiving, by one or more input devices, indications of one or more visual disturbances experienced by the patient during the preclinical evaluation procedure.

In some embodiments, flow diagram 1000 may further include controlling (e.g., via controller 650) a liquid lens (e.g., liquid lens 216, liquid lens 630) disposed distally to an artificial cornea and aligned with the optical axis, the liquid lens configured to introduce a plurality of defocus levels while the patient views the target. In some embodiments, for example, flow diagram 1000 may further include generating (e.g., via controller 650) a plurality of visual acuity curves corresponding to the plurality of defocus levels. In some embodiments, flow diagram 1000 may further include capturing images corresponding to a field of view of the patient while the patient views the target through the IOL assembly. In some embodiments, for example, flow diagram 1000 may further include correlating the captured images with patient inputs indicating the one or more visual disturbances.

In operation 1008, as shown in the example of FIGS. 1A–9, an assessment output is generated (e.g., via controller 650) based on the acquired patient response data, the assessment output characterizing visual performance associated with the IOL assembly (e.g., a visual acuity curve). In some embodiments, generating the assessment output may include generating a visual acuity curve corresponding to the IOL assembly. In some embodiments, the visual reference information and the assessment output may be based on received information identifying the IOL assembly, a vision assessment procedure, or both. In some embodiments, flow diagram 1000 may further include storing information indicative of one or more visual disturbances experienced by the patient while viewing the target through the model eye. In some embodiments, for example, the one or more visual disturbances comprises glare, halos, starbursts, reduced contrast, depth of focus artifacts, or a combination thereof.

In some embodiments, flow diagram 1000 may further include repeating operations 1002, 1004, 1006, 1008 for a second IOL assembly, where the second IOL assembly has at least one different optical characteristic than the IOL assembly. In some embodiments, for example, flow diagram 1000 may further include comparing assessment outputs between the IOL assembly and the second IOL assembly. In some embodiments, flow diagram 1000 may further include generating a baseline visual acuity curve for the patient prior to removably coupling the selected IOL assembly. In some embodiments, for example, flow diagram 1000 may further include comparing the baseline visual acuity curve with the at least one visual acuity curve corresponding to the selected IOL assembly.

It is to be understood that the phraseology or terminology herein is for the purpose of description and not of limitation, such that the terminology or phraseology of the present specification is to be interpreted by those skilled in relevant art(s) in light of the teachings herein.

The above examples are illustrative, but not limiting, of the embodiments of this disclosure. Other suitable modifications and adaptations of the variety of conditions and parameters normally encountered in the field, and which would be apparent to those skilled in the relevant art(s), are within the spirit and scope of the disclosure.

While specific embodiments have been described above, it will be appreciated that the embodiments may be practiced otherwise than as described. The description is not intended to limit the scope of the claims.

It is to be appreciated that the Detailed Description section, and not the Summary and Abstract sections, is intended to be used to interpret the claims. The Summary and Abstract sections may set forth one or more, but not all example embodiments as contemplated by the inventor(s), and thus, are not intended to limit the embodiments and the appended claims in any way.

The embodiments have been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments will so fully reveal the general nature of the embodiments that others can, by applying knowledge within the skill of the art, readily modify and/or adapt for various applications such specific embodiments, without undue experimentation, without departing from the general concept of the embodiments. Therefore, such adaptations and modifications are intended to be within the meaning and range of equivalents of the disclosed embodiments, based on the teaching and guidance presented herein.

The breadth and scope of the embodiments should not be limited by any of the above-described example embodiments, but should be defined only in accordance with the following claims and their equivalents.

The present disclosure may also be described in accordance with the following clauses:

Clause 1. An optical system comprising: a support structure; a model eye comprising: an artificial cornea coupled to the support structure and disposed along an optical axis of the optical system; and an intraocular lens (IOL) assembly configured to be removably coupled to the support structure, wherein in a coupled configuration the IOL assembly is disposed proximally to the artificial cornea and aligned with the optical axis; and an eyepiece assembly disposed proximally to the model eye and aligned with the optical axis.

Clause 2. The optical system of clause 1, further comprising: a liquid lens disposed distally to the artificial cornea and aligned with the optical axis; and a controller communicatively coupled to the liquid lens, the controller configured to transmit a control signal to control a shape of the liquid lens.

Clause 3. The optical system of clause 1 or clause 2, the IOL assembly comprising: a body defining: an exterior surface; a handle extending from the exterior surface; and an interior volume; and an IOL wet cell disposed within the interior volume.

Clause 4. The optical system of clause 3, wherein: the body further defines a recess from a distal surface; and the IOL assembly further comprises an aperture plate configured to be retained in the recess, the aperture plate aligned with the optical axis in a retained configuration.

Clause 5. The optical system of clause 3, the model eye further comprising: a base defining: a proximal end opening and a distal end opening, wherein the artificial cornea is disposed in the distal end opening; and a first circumferential opening configured to receive the IOL assembly into the base.

Clause 6. The optical system of clause 5, wherein: the base further defines a second circumferential opening; the model eye further comprises a first alignment component configured to be received into the second circumferential opening; and the IOL assembly is aligned with the optical axis when the exterior surface of the body contacts the first alignment component.

Clause 7. The optical system of clause 6, wherein: the optical system further comprises a second alignment component coupled to the support structure; and the artificial cornea is aligned with the optical axis when an exterior surface of the base contacts the second alignment component.

Clause 8. The optical system of clause 7, wherein the first alignment component contacts an upper surface of the second alignment component.

Clause 9. The optical system of clause 3, wherein: the IOL assembly further comprises an identifier; and the optical system further comprises a controller configured to: detect the identifier when the IOL assembly is in the coupled configuration; and apply, based on the identifier, one or more settings for a preclinical evaluation procedure that simulates vision of a patient after performing an IOL implantation.

Clause 10. The optical system of clause 9, wherein the controller is further configured to: receive, by one or more input devices, indications of visual disturbances experienced by the patient during the preclinical evaluation procedure.

Clause 11. The optical system of any one of clauses 1 to 10, wherein the eyepiece assembly comprises: a first lens; a beamsplitter disposed proximally to the first lens; and a second lens disposed proximally to the beamsplitter.

Clause 12. The optical system of clause 11, wherein: the optical system further comprises a viewfinder camera; and the beamsplitter is configured to reflect a portion of light incident on the model eye toward the viewfinder camera.

Clause 13. The optical system of clause 12, wherein: the optical system further comprises a pupil camera; and the beamsplitter is further configured to reflect a portion of light incident on the second lens toward the pupil camera.

Clause 14. The optical system of clause 13, wherein: the optical system further comprises an infrared light source oriented toward an eye of a patient; and the light incident on the second lens includes infrared light reflected from the eye.

Clause 15. The optical system of clause 14, further comprising: a controller configured to: track a position of a pupil of the eye; and generate, based on the position of the pupil, a visual acuity curve for the eye.

Clause 16. A method of simulating intraocular lens (IOL) implantation, the method comprising: configuring an optical simulation system by removably coupling an IOL assembly within a model eye aligned along an optical axis of the optical simulation system; positioning a patient to view a target through the model eye; presenting visual reference information as the target and acquiring patient response data while the patient views the target through the model eye; and generating an assessment output based on the acquired patient response data, the assessment output characterizing visual performance associated with the IOL assembly.

Clause 17. The method of clause 16, wherein removably coupling the IOL assembly comprises selecting one of a plurality of interchangeable IOL assemblies each providing a different optical characteristic.

Clause 18. The method of clause 16 or clause 17, wherein presenting the visual reference information comprises displaying optotypes, contrast patterns, defocus targets, simulated night driving scenes, or a combination thereof at a simulated distance.

Clause 19. The method of any one of clauses 16 to 18, wherein acquiring patient response data comprises receiving user input indicative of visual acuity, contrast sensitivity, subjective visual quality, or a combination thereof.

Clause 20. The method of any one of clauses 16 to 19, wherein generating the assessment output comprises generating a visual acuity curve corresponding to the IOL assembly.

Clause 21. The method of any one of clauses 16 to 20, further comprising storing information indicative of one or more visual disturbances experienced by the patient while viewing the target through the model eye.

Clause 22. The method of clause 21, wherein the one or more visual disturbances comprises glare, halos, starbursts, reduced contrast, depth of focus artifacts, or a combination thereof.

Clause 23. The method of any one of clauses 16 to 22, further comprising: repeating steps (1) through (4) of the method for a second IOL assembly, wherein the second IOL assembly has at least one different optical characteristic than the IOL assembly; and comparing assessment outputs between the IOL assembly and the second IOL assembly.

Clause 24. The method of any one of clauses 16 to 23, wherein the visual reference information and the assessment output are based on received information identifying the IOL assembly, a vision assessment procedure, or both.

Clause 25. A method of simulating intraocular lens (IOL) implantation, the method comprising: providing an optical system configured to simulate vision of a patient after an IOL implantation and having an optical axis, the optical system comprising: a support structure; a model eye coupled to the support structure, the model eye including an artificial cornea disposed along the optical axis and an IOL assembly configured to be removably coupled to the model eye such that, in a coupled configuration, the IOL assembly is disposed proximally to the artificial cornea and aligned with the optical axis; an eyepiece assembly disposed proximally to the model eye and aligned with the optical axis; and a controller communicatively coupled to at least one display and at least one input device; positioning the patient relative to the eyepiece assembly such that at least one eye of the patient is aligned with the optical axis to view a target through the model eye; removably coupling a selected IOL assembly to the model eye to configure one or more optical characteristics through which the patient views the target; receiving, by the controller, information indicative of at least one of (i) an identification of the selected IOL assembly, (ii) a type of vision assessment procedure to be performed, or (iii) a target distance or a display parameter associated with the target; determining, based at least in part on the received information, reference information to be presented on the at least one display as the target for assessing at least one aspect of the patient’s vision while the patient views the target through the selected IOL assembly; presenting, on the at least one display, the determined reference information as the target, and acquiring inputs indicative of patient responses to the presented reference information while the patient views the target through the selected IOL assembly; generating, by the controller and from the acquired inputs, at least one visual acuity curve corresponding to the selected IOL assembly; and storing, by the controller, information indicating one or more visual disturbances experienced by the patient while viewing the target through the selected IOL assembly.

Clause 26. The method of clause 25, further comprising: controlling, by the controller, a liquid lens disposed distally to the artificial cornea and aligned with the optical axis, the liquid lens configured to introduce a plurality of defocus levels while the patient views the target; and generating, by the controller, a plurality of visual acuity curves corresponding to the plurality of defocus levels.

Clause 27. The method of clause 25 or clause 26, wherein presenting the determined reference information comprises presenting the target at a plurality of distances from the model eye, and wherein the reference information is scaled or selected based on a distance at which the target is presented.

Clause 28. The method of any one of clauses 25 to 27, further comprising: tracking, by the controller, a position of a pupil of an eye of the patient; and generating, based on the position of the pupil, a visual acuity curve for the eye.

Clause 29. The method of any one of clauses 25 to 28, further comprising: capturing images of a pupil of an eye of the patient while the patient views the target; and validating inputs indicative of patient responses based on whether the pupil is aligned within a predefined range relative to the optical axis.

Clause 30. The method of clause 29, further comprising disregarding inputs indicative of patient responses acquired while the pupil is outside the predefined range.

Clause 31. The method of clause 29, further comprising providing an alert to the patient or an operator when the pupil is determined to be outside the predefined range.

Clause 32. The method of any one of clauses 25 to 31, further comprising: capturing images corresponding to a field of view of the patient while the patient views the target through the selected IOL assembly; and correlating the captured images with patient inputs indicating the one or more visual disturbances.

Clause 33. The method of any one of clauses 25 to 32, wherein: removably coupling the selected IOL assembly comprises selecting an aperture plate associated with a simulated lighting condition; and simulating at least one of photopic vision, mesopic vision, or scotopic vision while presenting the target.

Clause 34. The method of any one of clauses 25 to 33, wherein receiving information indicative of an identification of the selected IOL assembly comprises: detecting an identifier associated with the selected IOL assembly; and applying, based on the identifier, one or more settings for a preclinical evaluation procedure.

Clause 35. The method of clause 34, further comprising receiving, by one or more input devices, indications of one or more visual disturbances experienced by the patient during the preclinical evaluation procedure.

Clause 36. The method of any one of clauses 25 to 35, further comprising: generating a baseline visual acuity curve for the patient prior to removably coupling the selected IOL assembly; and comparing the baseline visual acuity curve with the at least one visual acuity curve corresponding to the selected IOL assembly

Clause 37. An intraocular lens (IOL) coin comprising: a body having an exterior surface and an interior volume; a wet cell disposed within the interior volume; and an IOL disposed within the wet cell.

Clause 38. The IOL coin of clause 37, further comprising a handle coupled to the exterior surface.

Clause 39. The IOL coin of clause 37 or clause 38, further comprising an aperture plate coupled to the body.

Clause 40. The IOL coin of any one of clauses 37 to 39, wherein the wet cell, the IOL, and the aperture plate are aligned along an optical axis of the IOL coin.

Clause 41. The IOL coin of any one of clauses 37 to 40, wherein the body further comprises an IOL alignment mechanism configured to adjust a position of the IOL to align with an optical axis of the IOL coin.

Clause 42. The IOL coin of any one of clauses 37 to 41, wherein the aperture plate is disposed distally to the wet cell.

What is claimed is:

1. An optical system comprising:
a support structure;
a model eye comprising:
an artificial cornea coupled to the support structure and disposed along an optical axis of the optical system; and
an intraocular lens (IOL) assembly configured to be removably coupled to the support structure, wherein in a coupled configuration the IOL assembly is disposed proximally to the artificial cornea and aligned with the optical axis; and
an eyepiece assembly disposed proximally to the model eye and aligned with the optical axis.

2. The optical system of claim 1, further comprising:
a liquid lens disposed distally to the artificial cornea and aligned with the optical axis; and
a controller communicatively coupled to the liquid lens, the controller configured to transmit a control signal to control a shape of the liquid lens.

3. The optical system of claim 1, the IOL assembly comprising:
a body defining:
an exterior surface;
a handle extending from the exterior surface; and
an interior volume; and
an IOL wet cell disposed within the interior volume.

4. The optical system of claim 3, wherein:
the body further defines a recess from a distal surface; and
the IOL assembly further comprises an aperture plate configured to be retained in the recess, the aperture plate aligned with the optical axis in a retained configuration.

5. The optical system of claim 3, the model eye further comprising:
a base defining:
a proximal end opening and a distal end opening, wherein the artificial cornea is disposed in the distal end opening; and
a first circumferential opening configured to receive the IOL assembly into the base.

6. The optical system of claim 5, wherein:
the base further defines a second circumferential opening;
the model eye further comprises a first alignment component configured to be received into the second circumferential opening; and
the IOL assembly is aligned with the optical axis when the exterior surface of the body contacts the first alignment component.

7. The optical system of claim 6, wherein:
the optical system further comprises a second alignment component coupled to the support structure; and
the artificial cornea is aligned with the optical axis when an exterior surface of the base contacts the second alignment component.

8. The optical system of claim 7, wherein the first alignment component contacts an upper surface of the second alignment component.

9. The optical system of claim 3, wherein:
the IOL assembly further comprises an identifier; and
the optical system further comprises a controller configured to:
detect the identifier when the IOL assembly is in the coupled configuration; and
apply, based on the identifier, one or more settings for a preclinical evaluation procedure that simulates vision of a patient after performing an IOL implantation.

10. The optical system of claim 9, wherein the controller is further configured to:
receive, by one or more input devices, indications of visual disturbances experienced by the patient during the preclinical evaluation procedure.

11. The optical system of claim 1, wherein the eyepiece assembly comprises:
a first lens;
a beamsplitter disposed proximally to the first lens; and
a second lens disposed proximally to the beamsplitter.

12. The optical system of claim 11, wherein:
the optical system further comprises a viewfinder camera; and
the beamsplitter is configured to reflect a portion of light incident on the model eye toward the viewfinder camera.

13. The optical system of claim 12, wherein:
the optical system further comprises a pupil camera; and
the beamsplitter is further configured to reflect a portion of light incident on the second lens toward the pupil camera.

14. The optical system of claim 13, wherein:
the optical system further comprises an infrared light source oriented toward an eye of a patient; and
the light incident on the second lens includes infrared light reflected from the eye.

15. The optical system of claim 14, further comprising:
a controller configured to:
track a position of a pupil of the eye; and
generate, based on the position of the pupil, a visual acuity curve for the eye.

16. A method of simulating intraocular lens (IOL) implantation, the method comprising:
configuring an optical simulation system by removably coupling an IOL assembly within a model eye aligned along an optical axis of the optical simulation system;
positioning a patient to view a target through the model eye;
presenting visual reference information as the target and acquiring patient response data while the patient views the target through the model eye; and
generating an assessment output based on the acquired patient response data, the assessment output characterizing visual performance associated with the IOL assembly.

17. The method of claim 16, wherein:
presenting the visual reference information comprises displaying optotypes, contrast patterns, defocus targets, simulated night driving scenes, or a combination thereof at a simulated distance; and
acquiring patient response data comprises receiving user input indicative of visual acuity, contrast sensitivity, subjective visual quality, or a combination thereof.

18. The method of claim 16, wherein generating the assessment output comprises generating a visual acuity curve corresponding to the IOL assembly.

19. The method of claim 16, further comprising storing information indicative of one or more visual disturbances experienced by the patient while viewing the target through the model eye.

20. The method of claim 16, further comprising:
repeating steps (1) through (4) of the method for a second IOL assembly, wherein the second IOL assembly has at least one different optical characteristic than the IOL assembly; and
comparing assessment outputs between the IOL assembly and the second IOL assembly.
